SEQUENCE LISTING (2 sequences)

SEQ ID NO: 1 - DNA, 24 bp, Artificial Sequence
  PCR primer
caaactgaagctcgcactctcgcc

SEQ ID NO: 2 - DNA, 24 bp, Artificial Sequence
  PCR Primer
gcaaagaccctcaaaacatcccag

(19) United States
(12) Patent Application Publication
(10) Pub. No.: US 2004/0180846 A1
(43) Pub. Date: Sep. 16, 2004

(54) Connexin enhances chemotherapy-induced apoptiosis in human cancer cells inhibiting tumor cell proliferation

(76) Inventors: Ruo-Pan Huang, Norcross, GA (US); Alton L. Boynton, Redmond, WA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 10/469,573

(22) PCT Filed: Mar. 1, 2002
(86) PCT No.: PCT/US02/06284

Related U.S. Application Data
(60) Provisional application No. 60/272,795, filed on Mar. 1, 2001.

(51) Int. Cl. A61K 48/00; A61K 39/395; A61K 9/127

(52) U.S. Cl. 514/44; 424/143.1; 424/93.2; 424/145.1; 514/27; 514/34; 514/449; 514/12; 424/450

(57) ABSTRACT

The present invention provides methods and compositions for the inhibition of proliferation rate of target cells, for example tumor cells. In particular, a nucleic acid encoding a connexin protein, fragment, derivative or analog thereof can be incorporated into a target cell. Expression of the nucleic acid sequence encoding the connexin protein, fragment, derivative or analog thereof, particularly connexin 43 and non-phosphorylated connexin 43, reduces the level of bcl-2 expression in the cells thereby inducing the cells to enter apoptosis. Connexin protein, fragments, derivatives, or analogs thereof can also be administered to the cell population to reduce bcl-2 expression inducing apoptosis in the cell population. It has further been found that the addition of an antagonist of MCP-1 activity can enhance the effects of connexin on tumor cell proliferation. Also, the prognosis of a subject undergoing standard chemotherapy can be assessed by correlating the expression levels of connexin and bcl-2.

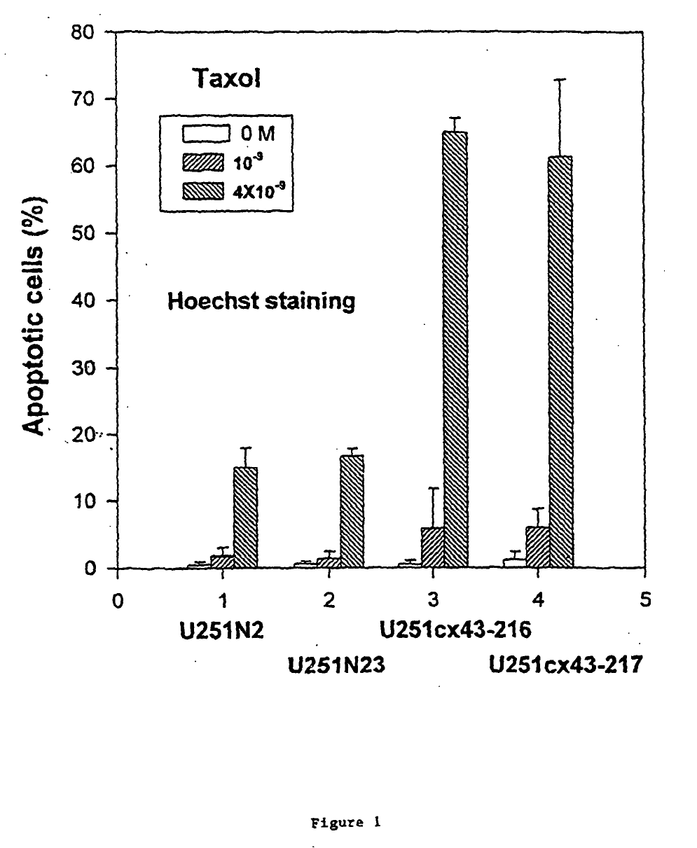

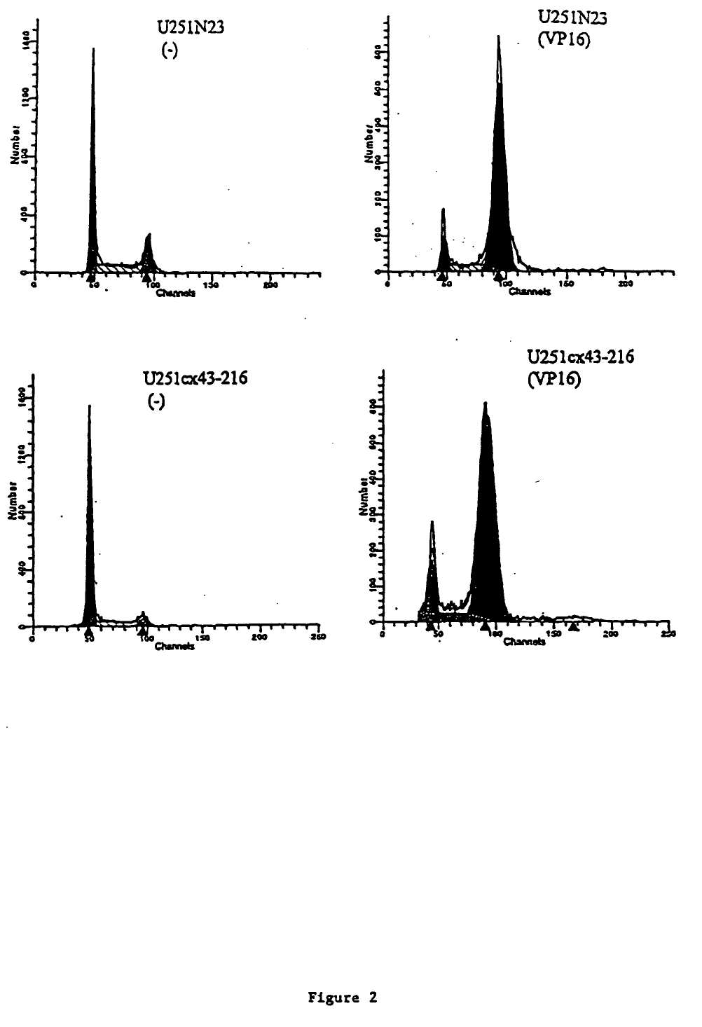

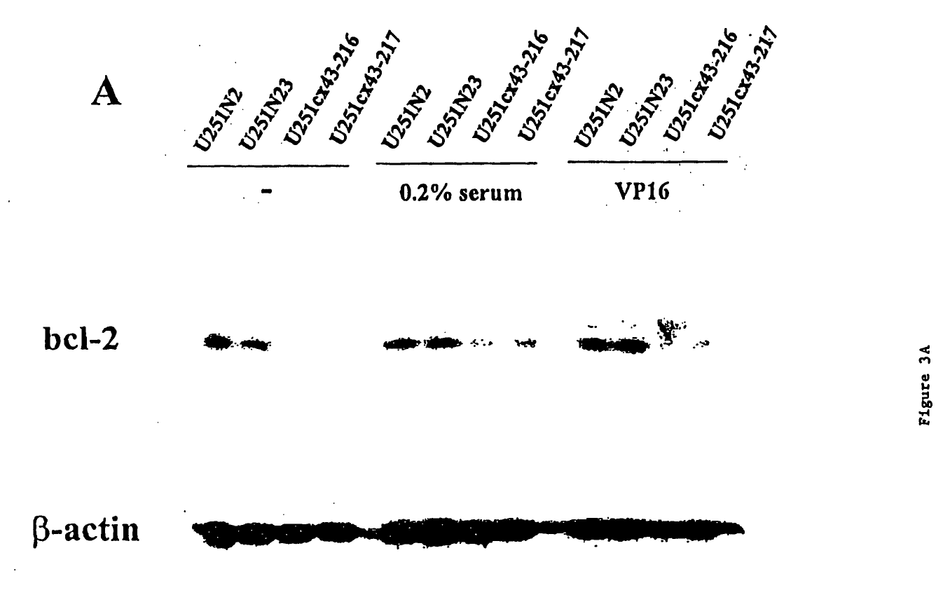

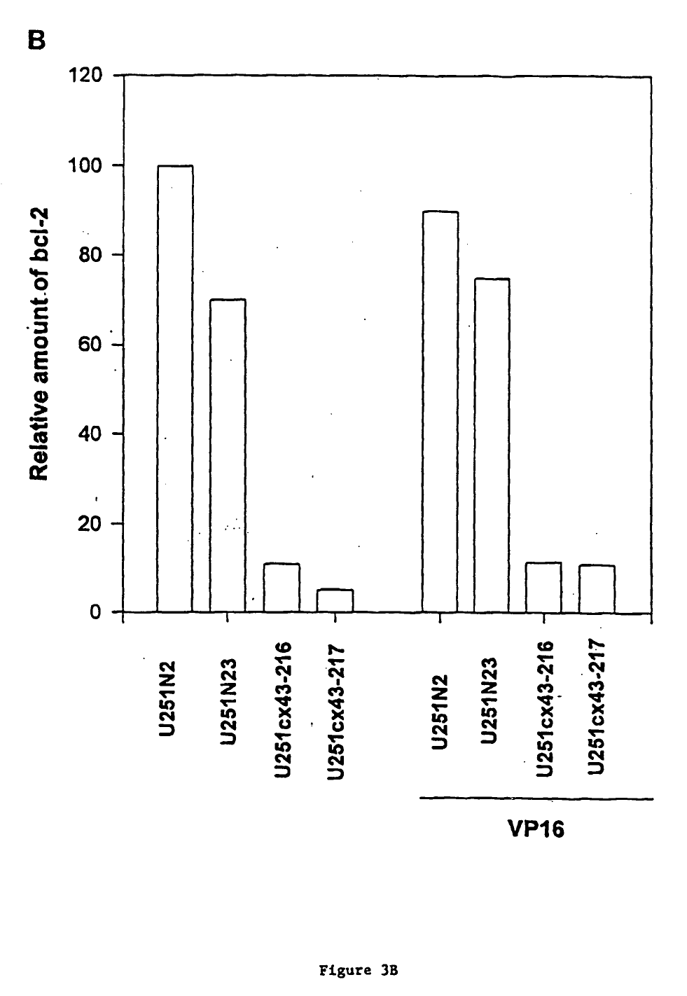

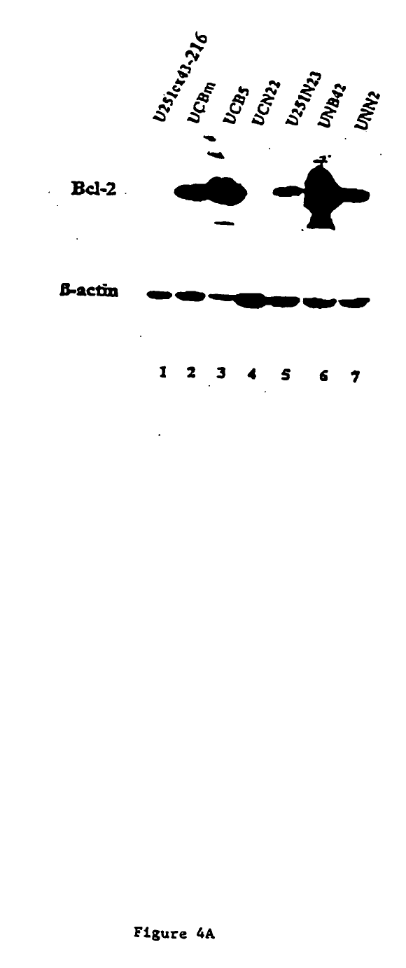

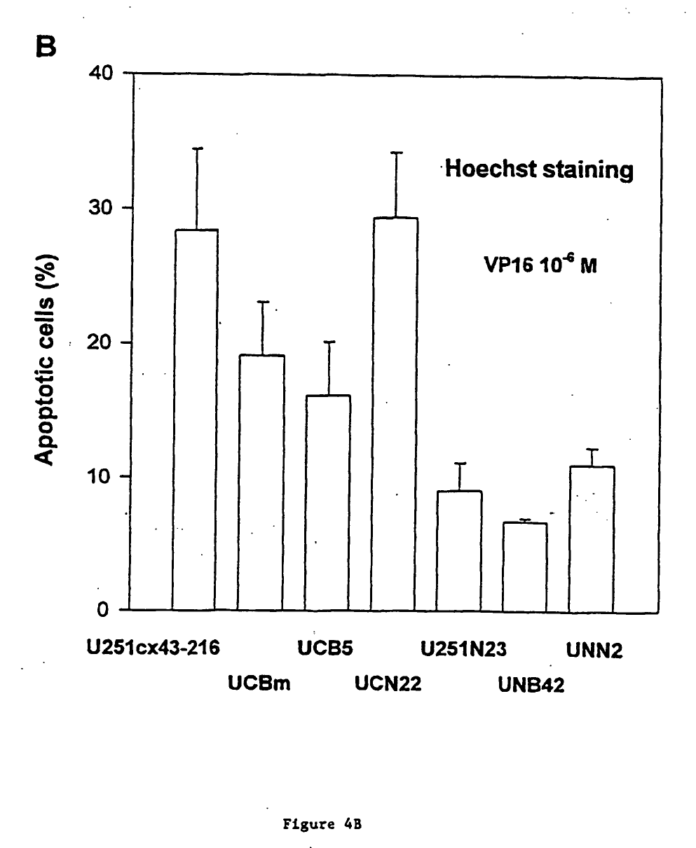

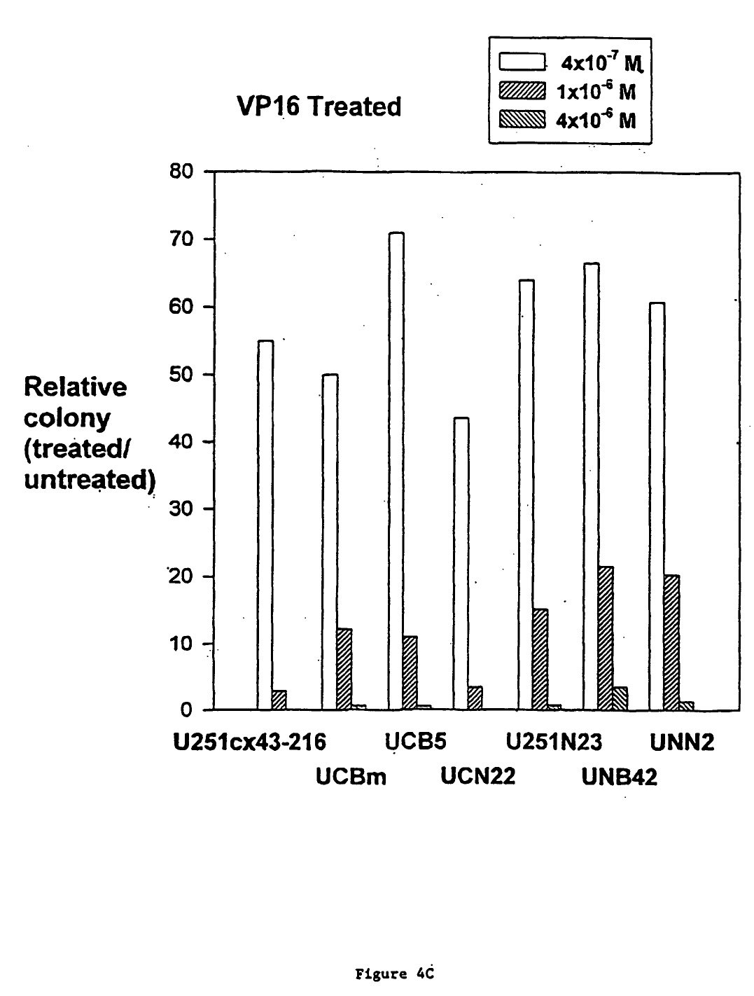

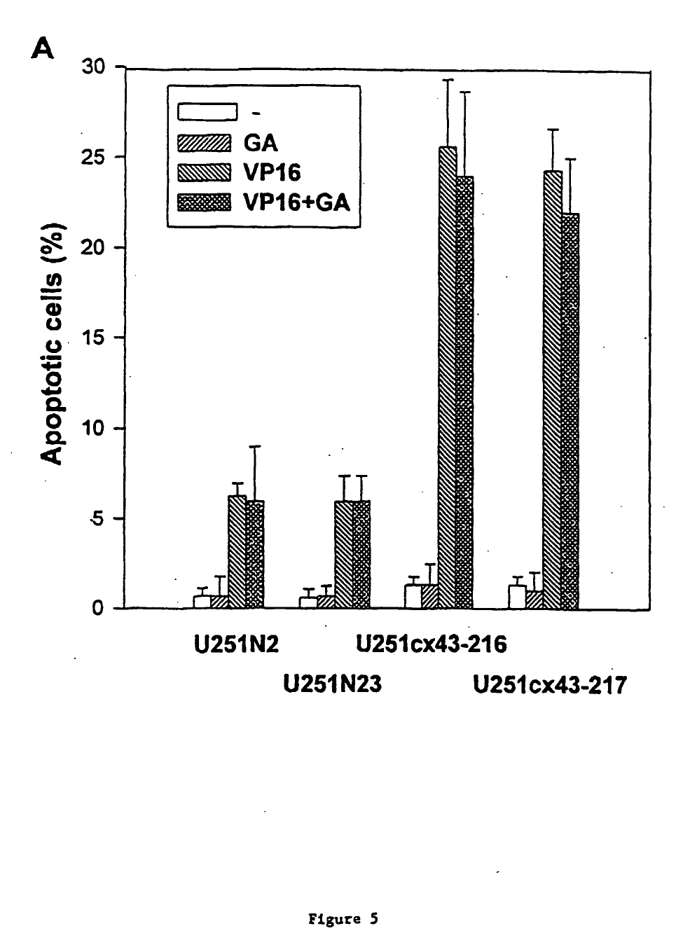

This application claims priority to U.S. provisional application serial No. 60/272,795, filed Mar. 1, 2001, the disclosure of which is incorporated herein by reference.

### STATEMENT AS TO RIGHTS TO INVENTIONS MADE UNDER FEDERALLY SPONSORED RESEARCH AND DEVELOPMENT

This work was supported by National Institutes of Health grants CA 39745 CA 58064 and CA 89273. The United States Government may have certain rights in the invention.

### BACKGROUND OF THE INVENTION

Adjacent cells can directly share ions and small molecules of less than 1,000 daltons in size through intercellular channels present in the morphological structure known as a gap junction (Trosko and Chang, Mutat. Res. 480-481:219-229 (2001); Yamasaki et al. Cancer Detect. Prev. 23:273-279 (1999); Yamasaki et al., C.R. Acad. Sci. III 322:151-159 (1999)). Gap junctions can be found in almost all mammalian tissues.

Gap junction communication (GJC) is believed to be involved in the regulation of cell homeostasis, cell proliferation, and cell differentiation. Accumulated evidence indicates that cx proteins may function as tumor suppressor genes. Many tumor-promoting agents, oncogenes and growth factors inhibit GJC (Yamasaki et al., Cancer Detect Prev. 23:273-279 (1999); Yamasaki et al., C.R. Acad. Sci. III 322:151-159 (1999)). In contrast, anti-neoplastic agents, such as retinoids, vitamin D and carstenoids up-regulate GJC (Trosko and Chang, Mutat. Res. 460-481:219-229 (2001); Yamasaki et al., Cancer Detect. Prev. 23:273-279 (1999)).

Connexin 43 (cx43) is a member of the gap junction (GJ) protein family, connexins (cxs), which consist of at least 15 homologous proteins ranging in size from 26 to 56 kilodaltons (kDa) (Yamasaki and Naus, Carcinogenesis 17:1199-1213 (1996)). These cxs are differentially expressed in a variety of tissues. Differential expression is generally believed to reflect cell specific regulation of gap junctional coupling and functional demands for gap junctions in different cell types. Cx43 is widely expressed, and like other gap junction proteins, forms intercellular plasma membrane channels that allow ions and small molecules of less than 1 kDa to pass through. Cx43 plays an important role in tissue homeostasis, embryonic development, cell proliferation and differentiation. Brain and heart tissues are found to particularly express cx43 (Yamasaki et al., Carcinogenesis 17:1199-1213 (1996)). Cx43 knockout mice die at birth due to cardiac malformations, suggesting a critical role of cx43 in development and in the fundamental physiology of multicellular organisms (Reaume et al., Science 267:1831-1834 (1995)).

Cx43 is a tumor suppressor gene (Chen et al., Cell Growth Differ. 6:681-6902 (1995); Huang et al., Cancer Res. 58:5089-5096 (1998); Yamasaki and Naus, Carcinogenesis 17:1199-1213 (1996)). Expression of cx43 is reduced in human mammary carcinoma (Lee et al., J. Cell Biol. 118:1213-1221 (1992); Tomasetto et al., J. Cell Biol. 122:157-167 (1993)), prostate cancer (Hossain et al., Prostate 38:55-59 (1999); Tsai et al., Biochem. Biophys. Res. Commun. 227:64-69 (1996); Wilgenbus et al., Int. J. Cancer 51:522-529 (1992)), human glioblastoma (Huang et al., Cancer Res. 58:5089-5096 (1998); Huang et al., J. Surg. Oncol. 70:21-24 (1999)), skin squanous-cell carcinoma (Sawey et al., Mol. Carcinog. 17:49-61 (1996)), lung cancer cells (Cesen-Cummings et al., Carcinogenesis 19:61-67 (1998); Jinn et al., Cancer Lett. 127:161-169 (1998); Zhang et al., Carcinogenesis 19:1889-1894 (1998)), esophageal cancer cells (Garber et al., Carcinogenesis 18:1149-1153 (1997); Oyamada et al., J. Cancer Res. Clin. Oncol. 120:445-453 (1994)) cervical cancer (King et al., Carcinogenesis 21:311-315 (2000), ovarian cancer (Hana et al., Carcinogenesis 20:1369-1376 (1999); (Ambauer et al., Am. J. Obstet. Gynecol. 182:999-1000 (2002)), uterine leiomyomata (Regidor et al., Gynecol. Endocrinol. 15:113-122 (2001)), endometrial cancer (Saito et al., Inst. J. Cancer 93:317-323 (2001)), and human mesothelioma (Pelin et al., Carcinogenesis 15:2673-2675 (1994)).

Transfection of cx43 restored GJC and several “normal” phenotypes to neoplastic cells, including rat C6 glioma (Naus et al., Cancer Res. 52:42084213 (1992); Zhu et al., Proc. Natl. Acad. Sci. USA 88:1883-1887 (1991)), human mammary carcinoma (cx26 and cx43) (Hirschi et al., Cell Growth Differ. 7:861-870 (1996)), human glioblastoma (Huang et al., Cancer Res. 52:4208-4213 (1998)), human hepatoma cells (cx32)(Eghbali et al., Proc. Natl. Acad. Sci. USA 80:10701-10705 (1991)), transformed dog kidney epithelial cells (cx43) (Chen et al., Cell Growth Differ. 6:681-690 (1995)), rhabdomyosarcoma cells (Proulx et al., Cell Growth Differ. 8:533-540 (1997)) and lung cancer cells (Zhang et al., Oncogene 20:4138-4149 (2001); Zhang et al., Carcinogenesis 19:1889-1894 (1998)). This was evidenced by reduced cell growth in vitro and/or decreased tumorigenicity in nude mice. In contrast, reduced expression of cx43 by transfection of anti-sense cDNA (Goldberg et al., Mol. Carcinogenesis 11: 106-114 (1994)) or by treatment of cells with anti-sense oligonucleotides (Ruch et al., Mol. Carcinogenesis 14:269-274 (1995)) resulted in abnormal growth regulation. Cell proliferation of fibroblasts from cx43-knockout mice was significantly increased compared to cells expressing cx43 (Martyn et al., Cell Growth Differ. 8:1015-1027 (1997)). Thus, direct and indirect evidence strongly supports an active role of cx43 in the maintenance of the non-neoplastic phenotype.

The mechanisms responsible for tumor suppression by cx43 are not fully characterized and may be through a different mechanism in different cell types. Expression of cx proteins restored differentiation potential in human mammary carcinoma cells (cx36 and cx43) (Hirochi et al., Cell Growth Differ. 7:861-870 (1996)) and induced myogenic differentiation in rhabdomyosarcoma cells (cx43) (Proul et al., Cell Growth Differ. 8:533-540 (1997)). Cx43 appears to inhibit proliferation of U205 cells by increasing the levels of p27 proteins via post-transcriptional regulatory mechanisms. (Zhang et al., Oncogene 20:4138-4149 (2001)). Transfection of the cx43 gene also enhanced genetic stability in HeLa cells (Zhu et al., Cancer Res. 57:2148-2150 (1997)). Cx43 may also be involved in the regulation of cell cycle progression (Chen et al., Cell Growth Differ. 6:681-690 (1995)). Suppression of rat glioma cell growth by cx43 may be due to regulation of a number of secreted factors. (Gapta et al., Mol. Pathol. 54:293-299 (2001); Goldberg et al., Cancer Res. 60:6018-6026 (2000); Naus et al., Brain Res. Rev. 32:259-266 (2000)).

Apoptosis, or programmed cell death, is a fundamental biological phenomenon that plays a crucial role in normal tissue homeostasis (Wyllie, Br. Med. Bull. 53:451-465 (1997)). Essentially all cytotoxic anti-cancer drugs as well as radiation commonly used in the treatment of human malignancies ultimately kill cancer cells primarily by inducing apoptosis and at least partially depend on the same biological mechanisms involved in physiological cell-death control. Therapeutic targeting to induce an increase in apoptosis in tumor cells will have a significant impact on the treatment of cancer (Reed, Semin. Hematol. 34(Suppl.5):9-19 (1997)).

A large body of experimental evidence suggests that apoptosis is regulated by both apoptosis blockers such as bcl-2, mcl-I, bag-1 and A1, and by apoptosis promoters such as bax-1, bak-1, bad-1, p53 and c-myc. Although its biochemical mechanisms remain enigmatic, the bcl-2 protein appears to control a distal step in what may represent a final common pathway for apoptotic cell death (Reed, Nature 387:773-776 (1997)).

The present invention provides additional compositions and methods for the reducing the proliferation of cancer cells in an individual. The compositions reduce the expression of bcl-2 increasing the apoptosis of tumor cells. The compositions and methods further increase the effectiveness of chemotherapeutic drugs by reducing the concentration of drug required to reduce the proliferation of, or to kill, cancer cells.

### SUMMARY OF THE INVENTION

The present invention provides methods for inhibiting the proliferation of tumor cells in a mammal by providing a nucleic acid sequence which encodes a connexin. The connexin can be connexin protein, an active fragment of connexin or derivatives or analogs thereof. The nucleic acid when provided to a target cell reduces the expression level of the anti-apoptotic protein bcl-2 which leads to cell death. The administration of a connexin nucleic acid enhances the sensitivity of certain target cells, i.e., glioblastoma cells, to chemotherapeutic agents, e.g., etoposide, paclitaxel, and doxorubicin, such that suboptimal levels of the drugs induces apoptosis in the cells. By reducing the expression level of bcl-2 more cells enter the cell death pathway, thereby inhibiting the proliferation of tumor cells in an animal, i.e., a mammal.

Connexin protein, polypeptide fragments, derivatives and analogs thereof can also be provided to the cell population to decrease bcl-2 expression in inducing apoptosis in the cells when combined with the chemotherapeutic agent. In addition the connexin protein, polypeptide fragments, derivatives and analogs thereof can be combined with antagonists of monocyte chemotactic protein-1 (MCP-1) activity with or without the chemotherapeutic agent. Moieties that can be used as MCP-1 antagonists can either prevent or reduce the expression of MCP-1 protein or can prevent or reduce the interaction of MCP-1 protein with its receptor.

The connexin protein, polypeptide fragment, derivative and analog thereof, of the invention can be connexin 26, connexin 32, connexin 43 or connexin 45. In a particular embodiment connexin 43 is used. The nucleic acids and proteins or peptides can be administered to a subject in a number of ways including, direct injection, microparticulate bombardment, liposomes, targeted liposomes, microparticles, microcapsules, or as complexes with a cell specific binding ligand. Further, a nucleic acid of the invention can be provided as part of a recombinant retrovirus, adeno-associated virus, HIV or Herpes virus. Therefore, various formulations and methods for the administration of the compositions of the present invention are provided.

Further, the invention provides methods for the diagnosis, and monitoring of disease prognosis and treatment. In a specific embodiment, the level of connexin expression and level of bcl-2 expression are determined and a poor prognosis is associated with a high level of bcl-2 expression and low level of connexin expression. A good prognosis can be associated with a high level of connexin expression and low bcl-2 expression level. A treatment regimen can be monitored by assaying the expression levels of bcl-2 and connexin and considering a change in treatment modality should the level of bcl-2 expression begin to increase and/or the level of connexin decrease.

### DESCRIPTION OF THE SPECIFIC EMBODIMENTS

The patents and publications cited in this disclosure reflect the level of skill in the art to which this invention pertains and are herein individually incorporated by reference for all purposes.

Unless defined otherwise, all technical and scientific terms used herein have the same meaning as commonly understood by one of ordinary skill in the art to which this invention belongs. Singleton et al., Dictionary of Microbiology and Molecular Biology, second edition, John Wiley and Sons, New York (1994), provides one of skill with a general dictionary of many of the terms used in this invention. Although any methods and materials similar or equivalent to those described herein can be used in the practice or testing of the present invention, typical methods and materials are described. For purposes of the present invention, the following terms are defined below.

Definitions

The term “tumor cell” or “cancer cell” or “neoplastic cell” denotes a cell that demonstrates inappropriate, unregulated proliferation. A “human” tumor is comprised of cells that have human chromosomes. Such tumors include those in a human patient, and tumors resulting from the introduction into a non-human host animal of a malignant cell line having human chromosomes into a non-human host animal.

“Non-tumorigenic cell” is a cell that is unable to form a tumor when introduced into a host organism. Examples include fibroblasts, epithelial cells, endothelial cells, bone cells, keratinocytes, and any cell that can be cultured in tissue culture, including tissue explants. Another kind of non-tumorigenic cells are cells that are normally tumorigenic but are treated to remove their tumorigenicity, for example, irradiated, engineered non-tumorigenic cells derived from tumors.

The phrase “inhibiting cell growth,” “inhibiting tumor growth,” “inhibition of proliferation,” or “inhibiting proliferation” generally means that the rate of increase in mass, size, number and/or the metabolism of treated cells and/or tumors is slower as a result of treatment than that of non-treated cells and/or tumors. The growth of a cell line or tumor is said to be “inhibited” by a treatment if, when assayed by means such as radioisotope incorporation into the cells, the treated cells increase in number at a rate that is less than the proliferation rate of untreated control cells, and typically less than about 50% of the untreated cell proliferation rate. In a particular embodiment, the growth rate is inhibited by at least 80%. If growth is assayed by a means such as plating in methylcellulose, the growth of a cell line is said to be “inhibited” if the treated cells give rise to less than the number of colonies that grow from a like number of untreated cells. Typically, the number of colonies from treated cells is less than about 70% of the number from untreated cells. In a particular embodiment, the number of colonies is decreased by at least 50%. “Inhibition of cell growth” also encompasses zero growth and, most importantly, consequent death of the tumor cells and eradication of the tumor. When measured in vivo, “inhibition of tumor growth” encompasses fewer or smaller tumors (for example, smaller diameter) as compared to control animals or untreated patients.

Inhibition can be evaluated by any accepted method of measuring whether growth or size of the tumor and/or increase in the number of cancerous or tumor cells has been slowed, stopped, or reversed. This includes direct observation and indirect evaluation such as subjective symptoms or objective signs. The clinician may notice a decrease in tumor size or tumor burden (number of tumors) based on physical exam, laboratory parameters, tumor markers, or radiographic findings. Alternatively, if the mammal is human, the patient may note improved vigor or vitality or decreased pain as subjective symptoms of improvement or response to therapy. Some laboratory signs that the clinician can observe for response to therapy include normalization of tests such as white blood cell count, red blood cell count, platelet count, erythrocyte sedimentation rate, and various enzyme levels such as transaminases and hydrogenases. Additionally, the clinician may observe a decrease in a detectable tumor marker such as D2-2 (U.S. Pat. No. 5,990,294, incorporated herein by reference), CXCR-4 (International Patent Publication WO 99/50461), CD44 (see e.g., Resnick et al., Mol. Diagn. 4:219-232 (1999)), IL13 receptor (see, for example, Debinski et al., Int. J. Oncol. 15:481-486 (1999) and EGF receptor (see e.g., Hunter et al., J. Neuropathol. Exp. Neurol. 54:57-64 (1995)) in glioblastoma, or chorioembryonic antigen (CEA), and the like. Alternatively, other tests can be used to evaluate objective improvement such as computerized axial tomography (CAT) scans, nuclear magnetic resonance (MRI) scans and positron emission testing (PET).

The term “nucleic acid” refers to deoxyribonucleotides or ribonucleotides, and polymers thereof in either single- or double-stranded form, and unless specifically limited, encompasses known analogues of natural nucleotides. Unless otherwise indicated, a particular nucleic acid sequence implicitly encompasses conservatively modified variants thereof and complementary sequences and as well as the sequence explicitly indicated.

The phrase “heterologous nucleic acid” generally denotes a nucleic acid that has been isolated, cloned and introduced into and/or expressed in a manner, cell or cellular environment other than the manner, cell or cellular environment in which the nucleic acid or protein may typically be found in nature. The term encompasses both nucleic acids originally obtained from a different organism or cell type than the cell type in which it is expressed, and also nucleic acids that are obtained from the same cell line as the cell line in which it is expressed.

The phrase “a nucleic acid sequence encoding” refers to a nucleic acid which contains sequence information that, if translated, yields the primary amino acid sequence of a specific protein or peptide. This phrase specifically encompasses degenerate codons (i.e., different codons which encode a single amino acid) of the native sequence or sequences which may be introduced to conform with codon preference in a specific host cell.

The term “recombinant” or “engineered” when used with reference to a cell indicates that the cell replicates or expresses a nucleic acid or expresses a peptide or protein encoded by a nucleic acid, whose origin is exogenous to the cell. Recombinant cells can express nucleic acids that are not found within the native (non-recombinant) form of the cell. Recombinant cells can also express nucleic acids natively expressed in the cell, wherein the nucleic acids are reintroduced into the cell by artificial means in order to alter the expression of that gene.

The term “connexin” denotes a family of genes and gene products wherein the gene products are structural subunits of gap junctions, and variants thereof. “Connexin” further denotes nucleic acid sequences and their gene products, wherein the gene products are recognized by antibodies that specifically bind to a connexin protein and, when expressed in cells, may be present in gap junctions. For a discussion of the connexin family of proteins, see Beyer et al., J. Membr. Biol. 116:187-194 (1990), and references cited therein.

The term “connexin protein” denotes a protein, or fragments thereof, that forms part of the physical structure of a connexin. The connexin is, for example, connexin 26 (Lee et al.; J. Cell Biol. 118:1213-1221 (1992), 32 (Kumor and Gilula J. Cell Biol. 103:767-776 (1986), 43 (Fishman et al., J. Cell Biol. 111:589-598 (1990), or 45 (Kanter et al., J. Mol. Cell. Cardiol. 26:861-868 (1994), the sequences of these proteins have been published and the references cited herein incorporated by reference. Connexin as used herein also refers to fragments or portions of the connexin protein which are capable of inhibiting the expression of bcl-2 and/or increasing the sensitivity of a glioblastoma cell line to sub-optimal levels of a chemotherapeutic drug.

The phrase “heterologous nucleic acid that encodes a connexin, derivative or fragment thereof, refers to those molecules that actively functions as a connexin which can modulate the expression of bcl-2 in a transfected cell resulting in a decrease in the rate of proliferation of the cells. One measure of this effect is a resulting increased apoptosis and a reduction in cell proliferation with sub-optimal concentrations of a chemotherapeutic drug, i.e., paclitaxel, etoposide or doxorubicin, and the like. A “sub-optimal” concentration of a drug is used herein to denote that concentration of a particular drug which is below the IC₅₀ for that drug.

Monocyte chemotactic protein-1 (MCP-1) is a member of the CC chemokine family (Cusing et al., Proc. Nat'l. Acad. Sci. USA 87:5134-5138 (1990); Koch et al., J. Clin. Invest. 90:792-779 (1990)). MCP-1 is known to be chemotactic for monocytes, T lymphocytes, basophils, and NK cells. Inhibition of MCP-1 activity has been shown to inhibit tumor metastasis and potentially to prolong survival of a subject with a tumor (WO 01/89565, incorporated herein by reference). As used herein an “antagonist” of MCP-1 activity can be any molecule that reduces the activity of MCP-1 by any biological mechanism. For example, an MCP-1 antagonist may bind either to MCP-1 or to an MCP-1 receptor, inhibiting the MCP-1/MCP-1 receptor interaction. MCP-1 activity can be inhibited by decreasing the amount of MCP-1 (or MCP-1 receptor) protein and/or nucleic acid, by, e.g., increasing degradation of MCP-1 protein, MCP-1 mRNA, MCP-1 receptor protein or MCP-1 receptor mRNA. An increase in degradation may be specific, for example using a MCP-1 antagonist that specifically binds and targets an MCP-1 or MCP-1 receptor polypeptide or nucleic acid for destruction, or non-specific, for example, by generally increasing protein or mRNA turnover. An MCP-1 antagonist can also inhibit MCP-1 activity by decreasing transcription of an MCP-1 or MCP-1 gene and/or translation of MCP-1 or MCP-1 receptor mRNA into an MCP-1 or MCP-1 receptor polypeptide.

By “antagonize or “inhibit” is meant a decrease in the MCP-1 activity by at least 10%, more typically by at least 20% or 30%, and more typically by at lease 70%, 80% or 90%. MCP-1 activity in a biological sample can be measured by using one or the numerous techniques known in the art. For example, the relative amount of MCP-1 can be measured using a receptor assay, chemotaxis assay, tumor metastasis assay, tumor survival assay known in the art. The relative level of MCP-1 activity can also be measured by determining the level of MCP-1 mRNA, the level of MCP-1 protein, the activity of a reporter gene under the transcriptional control of a MCP-1 transcriptional regulatory region, or detecting the level or amount of specific interaction between MCP-1 with another molecule.

The antagonists of MCP-1 can be any type of molecule capable of interacting with MCP-1, MCP-1 nucleic acid, an MCP-1 receptor, or an MCP-1 receptor nucleic acid in a way that inhibits or antagonizes MCP-1 activity. For example, an MCP-1 antagonist can be an antibody, e.g., but not limited to, a polyclonal or monoclonal antibody, a bispecific antibody, a chimeric antibody, a single chain antibody or any antigen binding fragment or derivative thereof (Fab, F(ab)′₂, and the like, or a ligand, e.g., but not limited to, a peptide or small molecule. An MCP-1 antagonist can also be a functional nucleic acid, i.e., but not limited to, an antisense molecule, an aptamer, a ribozyme or catalytic nucleic acid, or a triplex forming molecule. The antisensce, and triplex forming molecules are designed to target MCP-1 or MCP-1 receptor nucleic acid. while the aptamers and catalytic nucleic acid molecules are designed to target either MCP-1 polypeptide, MCP-1 nucleic acid, MCP-1 receptor polypeptide, or MCP-1 receptor nucleic acid.

A “chemotherapeutic drug” as used herein refers to those drugs commonly used in the treatment of cancer. These agents act through an apoptotic mechanism of cell death. Each of the drugs can differ in the mechanism by which the cells enter apoptosis. “Apoptosis” refers to a regulated network of biochemical events which lead to a selective form of cell suicide, and is characterized by readily observable morphological and biochemical phenomena, such as fragmentation of the deoxyribonucleic acid (DNA), condensation of the chromatin, which may or may not be associated with endonuclease activity, chromosome migration in cell nuclei, the formation of apoptotic bodies, mitochondrial swelling, and the like. The cells of a tumor, such as for example, a carcinoma, a sarcoma, lymphoma, leukemia or melanoma, in an animal, i.e., a mammal, demonstrate a inhibition in the rate of proliferation.

The phrase “effective amount” means a dosage of a drug or agent sufficient to produce a desired result. The desired result can be subjective or objective improvement in the recipient of the dosage, a decrease in tumor size, a decrease in the rate of growth of cancer cells, a decrease in metastasis, or any combination of the above.

General Methods for Introduction of Connexin Protein or Selected Genes into Cells.

An important aspect of the present invention is a method for introducing connexin proteins and/or selected genes (e.g., a connexin protein, derivatives or fragments thereof, including a non-phosphorylated connexin) into cells. Standard eukaryotic transduction methods are used to produce cell lines which express connexin protein and, optionally, a drug resistance gene. It is expected that those of skill in the art are knowledgeable in the numerous systems available for transferring, cloning and expressing nucleic acids.

Briefly, the expression of natural or synthetic nucleic acids is typically achieved by operably linking a nucleic acid of interest (e.g., one encoding a connexin) to a promoter (which is either constitutive or inducible) and incorporating the construct into an expression vector. The vectors are suitable for replication and/or expression in prokaryotes, eukaryotes, or preferably both. Typical cloning vectors contain transcription and translation terminators, transcription and translation initiation sequences, and promoters useful for regulation of the expression of the particular nucleic acid. The vectors optionally comprise expression cassettes containing at least one independent terminator sequence, sequences permitting replication of the cassette in eukaryotes, or prokaryotes, or both, (e.g., shuttle vectors) and selection markers for both prokaryotic and eukaryotic systems. See Giliman and Smith, Gene 8:81-97 (1979); Roberts et al., Nature 328:731-734 (1987); Berger and Kimmel, Methods Enzymol., Vol. 152, Academic Press, Inc., San Diego, Calif. (Berger); Sambrook et al., Molecular Cloning: A Laboratory Manual (2nd ed.) Vol. 1-3, Cold Spring Harbor Laboratory, Cold Spring Harbor Press, NY (1989), (Sambrook); and Ausubel, F. M. et al., Current Protocols in Molecular Biology, eds., Current Protocols, a joint venture between Greene Publishing Associates, Inc. and John Wiley & Sons, Inc., (1994 Supplement) (Ausubel). Product information from manufacturers of biological reagents and experimental equipment also provide information useful in known biological methods. Such manufacturers include the SIGMA Chemical Company (Saint Louis, Mo.), R&D Systems (Minneapolis, Minn.), Pharmacia LKB Biotechnology (Piscataway, N.J.), CLONTECH Laboratories, Inc. (Palo Alto, Calif.), Chem Genes Corp., Aldrich Chemical Company (Milwaukee, Wis.), Glen Research, Inc., GIBCO BRL Life Technologies, Inc. (Gaithersburg, Md.), Fluka Chemica-Biochemika Analytika (Fluka Chemie AG, Buchs, Switzerland), and Applied Biosystems (Foster City, Calif.), as well as many other commercial sources known to one of skill.

The expression vector typically comprises a prokaryotic replicon covalently linked to an eukaryotic transcription unit or expression cassette that contains all the elements required for the expression of exogenous connexin protein in eukaryotic cells. A typical expression cassette contains a promoter linked to the DNA sequence encoding the selected connexin protein and signals required for efficient polyadenylation of the transcript.

Eukaryotic promoters typically contain at least two types of regulatory sequences, the TATA box and upstream promoter elements. The TATA box, located 25-30 base pairs upstream of the transcription initiation site, is thought to be involved in directing RNA polymerase to begin RNA synthesis. The other upstream promoter elements may determine the rate at which transcription is initiated.

Enhancer elements can stimulate transcription up to 1,000 fold from linked homologous or heterologous promoters. Enhancers are active when placed downstream or upstream from the transcription initiation site. Many enhancer elements derived from viruses have a broad host range and are active in a variety of tissues. For example, the SV40 early gene enhancer is suitable for many cell types. Other enhancer/promoter combinations that are suitable for the present invention include those derived from polyoma virus, human or murine cytomegalovirus, the long term repeat from various retroviruses such as murine leukemia virus, murine or Rous sarcoma virus and HIV. See, for example, Enhancers and Eukaryotic Expression, Cold Spring Harbor Press, Cold Spring Harbor, N.Y. (1983), which is incorporated herein by reference.

In the construction of the expression cassette, the promoter is typically positioned about the same distance from the heterologous transcription start site as it is from the transcription start site in its natural setting. As is known in the art, however, some variation in this distance can be accommodated without loss of promoter function.

In addition to a promoter sequence, the expression cassette should also contain a transcription termination region downstream of the connexin protein structural gene to provide for efficient termination. The termination region may be obtained from the same source as the promoter sequence or may be obtained from a different source.

If the mRNA encoded by the connexin protein structural gene is to be efficiently translated, polyadenylation sequences are also commonly added to the vector construct. Two distinct sequence elements are required for accurate and efficient polyadenylation: GU or U rich sequences located downstream from the polyadenylation site and a highly conserved sequence of six nucleotides, AAUAAA, located about 11-30 nucleotides upstream. Termination and polyadenylation signals that are suitable for the present invention include those derived from SV40, or a partial genomic copy of a gene already resident on the expression vector.

In addition to the elements already described, the expression vector of the present invention can typically contain other specialized elements intended to increase the level of expression of cloned nucleic acids or to facilitate the identification of cells that carry the transduced DNA. For instance, a number of animal viruses contain DNA sequences that promote the extra chromosomal replication of the viral genome in permissive cell types. Plasmids bearing these viral replicons are replicated episomally as long as the appropriate factors are provided by genes either carried on the plasmid or with the genome of the host cell.

The vector may or may not comprise a eukaryotic replicon. If a eukaryotic replicon is present, then the vector can be amplifiable in eukaryotic cells using the appropriate selectable marker. If the vector does not comprise a eukaryotic replicon, no episomal amplification is possible. Instead, the transduced DNA integrates into the genome of the engineered cell, where the promoter directs expression of the desired nucleic acid.

The vectors can include selectable markers which can be used for nucleic acid amplification such as the sodium, potassium ATPase, thymidine kinase, aminoglycoside phosphotransferase, hygromycin B phosphotransferase, xanthine-guanine phosphoribosyl transferase, CAD (carbamyl phosphate synthetase, aspartate transcarbamylase, and dihydroorotase), adenosine deaminase, dihydrofolate reductase, and asparagine synthetase and ouabain selection. Alternatively, high yield expression systems not involving nucleic acid amplification are also suitable, such as using a bacculovirus vector in insect cells, with connexin protein encoding sequence under the direction of the polyhedrin promoter or other strong bacculovirus promoters.

The expression vectors of the present invention will typically contain both prokaryotic sequences that facilitate the cloning of the vector in bacteria as well as one or more eukaryotic transcription units that are expressed only in eukaryotic cells, such as mammalian cells. The prokaryotic sequences are preferably chosen such that they do not interfere with the replication of the DNA in eukaryotic cells.

Once a nucleic acid is synthesized or isolated and inserted into a vector and cloned, one may express the nucleic acid in a variety of recombinantly engineered cells known to those of skill in the art. Expression of a an exogenous nucleic acid can be enhanced by including multiple copies of, for example, a connexin protein-encoding nucleic acid in an engineered cell, by selecting a vector known to reproduce in the host, thereby producing large quantities of protein from exogenous inserted DNA (such as, pUC8, ptac12, or pIN-III-ompA1, 2, or 3, and the like), or by any other known means of enhancing peptide expression. Connexin protein molecules will be expressed when the DNA sequence is functionally inserted into a vector. “Functionally inserted” means that it is inserted in proper reading frame and orientation and operably linked to proper regulatory elements. Typically, a connexin protein gene will be inserted downstream from a promoter and will be followed by a stop codon. However, production as a hybrid protein optionally followed by cleavage may be used, if desired.

Vectors for Introduction and Expression of Connexin Protein in Cells.

Vectors to which connexin protein-encoding nucleic acids are operably linked can be used to introduce these nucleic acids into host cells and mediate their replication and/or expression. “Cloning vectors” are useful for replicating and amplifying the foreign nucleic acids and obtaining clones of specific foreign nucleic acid-containing vectors. “Expression vectors” mediate the expression of the foreign nucleic acid. Some vectors are both cloning and expression vectors.

In general, the particular eukaryotic expression vector used to transport connexin protein or any other gene into the cell may not be particularly critical. Any of the conventional vectors used for expression in eukaryotic cells may be used. Expression vectors containing regulatory elements from eukaryotic viruses such as retroviruses are typically used. SV40 vectors include pSVT7 and pMT2. Vectors derived from bovine papilloma virus include pBV-1MTHA, and vectors derived from Epstein Bar virus include pHEBO, p2O5, and the like. Other exemplary vectors include pMSG, pAV009/A.sup.⁺, pMTO10/A.sup.30, pMAMneo-5, bacculovirus pDSVE, and any other vector allowing expression of proteins under the direction of promoters derived from the SV40 early promoter, SV40 later promoter, metallothionein promoter, murine mammary tumor virus promoter, Rous sarcoma virus promoter, polyhedrin promoter, cytomegalovirus promoter, or other promoters shown effective for expression in eukaryotic cells.

While a variety of vectors may be used, it should be noted that retroviral vectors are widely used for modifying eukaryotic cells in vitro because of the high efficiency with which the retroviral vectors transfect target cells and integrate into the target cell genome. Additionally, retroviral vectors are capable of infecting cells from a wide variety of tissues.

Retroviral vectors are produced by genetically manipulating retroviruses. Retroviruses are RNA viruses because the viral genome is RNA. Upon infection, this genomic RNA is reverse transcribed into a DNA copy which is integrated into the chromosomal DNA of transfected cells with a high degree of stability and efficiency. The integrated DNA copy is referred to as a pro-virus and is inherited by daughter cells as is any other gene. The wild type retroviral genome and the pro-viral DNA have three genes: the gag, the pol and the env genes, which are flanked by two long terminal repeat (LTR) sequences. The gag gene encodes the internal structural (nucleocapsid) proteins; the pol gene encodes the RNA directed DNA polymerase (reverse transcriptase); and the env gene encodes viral envelope glycoproteins. The 5′ and 3′ LTRs serve to promote transcription and polyadenylation of virion RNAs. Adjacent to the 5′ LTR are sequences necessary for reverse transcription of the genome (the tRNA primer binding site) and for efficient encapsulation of viral RNA into particles (the Psi site). See Mulligan, In: Experimental Manipulation of Gene Expression, Inouye (ed), 155-173 (1983); Mann et al., Cell 33:153-159 (1983); Cone and Mulligan, Proc. Natl. Acad. Sci. USA 81:6349-6353 (1984).

The design of retroviral vectors is well known to one of skill in the art. See Singer and Berg, supra. In brief, if the sequences necessary for encapsidation (or packaging of retroviral RNA into infectious virions) are missing from the viral genome, the result is a cis-acting defect which prevents encapsidation of genomic RNA. However, the resulting mutant is still capable of directing the synthesis of all virion proteins. Retroviral genomes from which these sequences have been deleted, as well as cell lines containing the mutant genome stably integrated into the chromosome are well known in the art and are used to construct retroviral vectors. Preparation of retroviral vectors and their uses are described in many publications including European Patent Application (EPA) 0 178 220, U.S. Pat. No. 4,405,712, Gilboa, Biotechniques 4:504-512 (1986), Mann et al., Cell 33:153-159 (1983), Cone and Mulligan, Proc. Natl. Acad. Sci. USA 81:6349-6353 (1984), Eglitis et al., Biotechniques, 6:608-614 (1988), Miller et al., Biotechniques 7:981-990 (1989), Miller Nature, supra (1992), Mulligan, supra (1993), and International Patent Application No. WO 92/07943. All of which are incorporated herein by reference.

Recombinant retroviral vectors useful in the present invention are prepared by inserting a nucleic acid encoding a connexin protein into a retrovirus vector and packaging the vector with retroviral capsid proteins by use of a packaging cell line. A packaging cell line is a genetically constructed mammalian tissue culture cell line that produces the necessary viral structural proteins required for packaging, but which itself is incapable of producing infectious virions. On the other hand, retroviral vectors used in conjunction with packaging cell lines lack sequences that encode viral structural proteins but retain the nucleic acid sequences necessary for packaging. To prepare a packaging cell line, an infectious clone of a desired retrovirus, in which the packaging site has been deleted, is constructed. Cells comprising this construct will express all structural proteins but the introduced DNA will be incapable of being packaged. Alternatively, packaging cell lines can be produced by transducing a cell line with one or more expression plasmids encoding the appropriate core and envelope proteins. In these cells, the gag, pol, and env genes can be derived from the same or different retroviruses.

A number of packaging cell lines suitable for the present invention are available in the art. Examples of these cell lines include Crip, GPE86, PA317, PG13, and the like. See Miller et al., J. Virol. 65:2220-2224 (1991), which is incorporated herein by reference. Examples of other packaging cell lines are described in Cone and Mulligan, Proc. Natl. Acad. Sci. USA 81:6349-6353 (1984), and in Danos and Mulligan, Proc. Natl. Acad. Sci. USA 85:6460-6464 (1988), Eglitis et al., Biotechniques, 6:608-614 (1988), Miller et al., Biotechniques 7:981-990 (1989), also all incorporated herein by reference. Amphotropic or xenotropic envelope proteins, such as those produced by PA317 and GPX packaging cell lines may also be used to package the retroviral vectors.

The resultant retroviral vector particle is generally incapable of replication in the host cell but is capable of integrating into the host cell genome as a pro-viral sequence containing the selected nucleic acid. As a result, engineered cells that contain the integrated recombinant vector are capable of producing the selected connexin protein.

In addition to the retroviral vectors mentioned above, cells can be infected or transfected with other eukaryotic vectors, including viral vectors such as adenoviral or adeno-associated viral vectors. See, e.g., Methods Enzymol. Vol. 185, Academic Press, Inc., San Diego, Calif. (Goeddel, ed.) (1990) or Krieger, Gene Transfer and Expression—A Laboratory Manual, Stockton Press, New York, N.Y. (1990), and the references cited therein. Adeno associated viruses (AAVs) require helper viruses such as adenovirus or herpes virus to achieve productive infection. In the absence of helper virus functions, AAV integrates (site-specifically) into a host cell's genome, but the integrated AAV genome has no pathogenic effect. The integration step allows the AAV genome to remain genetically intact until the host is exposed to the appropriate environmental conditions (e.g., a lytic helper virus), whereupon it re-enters the lytic life-cycle. Other AAV vectors may not integrate. Samulski, Curr. Op. Genet. Dev. 3:74-80 (1993), and the references cited therein provides an overview of the AAV life cycle. See also West et al., Virology 160:3847 (1987); Carter et al., U.S. Pat. No. 4,797,368 (1989); Carter et al., WO 93/24641 (1993); Kotin, Hum. Gene Therapy 5:793-801 (1994); Muzyczka, J. Clin. Invest. 94:1351 (1994), and Samulski, supra for an overview of AAV vectors.

Plasmids designed for producing recombinant vaccinia, such as pGS62, (Langford et al., Mol. Cell. Biol. 6:3191-3199 (1986)) can also be used. This plasmid consists of a cloning site for insertion of foreign nucleic acids, the P7.5 promoter of vaccinia to direct synthesis of the inserted nucleic acid, and the vaccinia tk gene flanking both ends of the foreign nucleic acid.

Transduction of Nucleic Acids into Cells.

There are several well-known methods of introducing nucleic acids into animal cells, any of which may be used in the present invention. These include: calcium phosphate precipitation, fusion of the recipient cells with bacterial protoplasts containing the DNA, treatment of the recipient cells with liposomes containing the DNA, DEAE dextran, receptor-mediated endocytosis, electroporation, micro-injection of the DNA directly into the cells, infection with viral vectors, and the like.

The methods of the present invention can be practiced in a variety of hosts. Typical hosts include mammalian species, such as humans, non-human primates, dogs, cats, cattle, horses, sheep, and the like. The amount of vector administered will depend upon the particular nucleic acid used, the mode of administration, the disease state being diagnosed; the age, weight, and condition of the patient and the judgment of the clinician; but will generally be between about 0.01 and about 50 mg per kilogram of body weight; preferably between about 0.1 and about 5 mg/kg of body weight or about 10⁸-10¹⁰ vectors per injection.

Connexin Polypeptides, Fragments, Derivatives and Analogs.

The invention further relates to connexin polypeptides, fragments, derivatives and analogs thereof. In one aspect, the invention provides amino acid sequences of connexin polypeptide, typically human connexin 43 polypeptide. In particular aspects, the polypeptides, fragments, derivatives, or analogs of connexin polypeptides are from an animal (e.g., human, mouse, rat, pig, cow, dog, monkey, and the like). The production and use of connexin polypeptides, fragments, derivatives and analogs thereof are also within the scope of the present invention. In a specific embodiment, the fragment, derivative or analog is functionally active (i.e., capable of exhibiting one or more functional activities associated with a full-length, wild-type connexin polypeptide). As one example, such fragments, derivatives or analogs which have the desired modulatory effect on the expression of bcl-2 and/or the ability to increase the sensitivity of target cells to the effects of a chemotherapeutic drug to inhibit proliferation can be used, for example, in in vitro cell culture assays, for reduction of bcl-2 expression, and the like. Fragments, derivatives or analogs that retain, or alternatively lack or inhibit, a desired connexin property of interest (e.g., increase in apoptosis in response to a chemotherapeutic drug, decrease in the production of bcl-2 mRNA, or modulation (e.g., inhibition or stimulation of cell proliferation) can be used as inducers, or inhibitors of such property and its physiological correlates. A specific embodiment relates to a connexin 43 fragment that can reduce bcl-2 expression and increase the sensitivity of glioblastoma cells to a chemotherapeutic agent. Fragments, derivatives or analogs of connexin can be tested for the desired activity by procedures known in the art, including but not limited to the functional assays described herein.

Connexin polypeptide derivatives include naturally-occurring amino acid sequence variants as well as those altered by substitution, addition or deletion of one or more amino acid residues that provide for functionally active molecules. Connexin polypeptide derivatives include, but are not limited to, those containing as a primary amino acid sequence of all or part of the amino acid sequence of a connexin polypeptide including altered sequences in which one or more functionally equivalent amino acid residues (e.g., a conservative substitution) are substituted for residues within the sequence, resulting in a silent change.

In another aspect, connexin polypeptides include those peptides having one or more consensus amino acid sequences shared by all connexin family members, but not found in other proteins. Connexin family members, including connexin 43 polypeptides, fragments, derivatives and/or analogs comprising one or more of these consensus sequences determined to be active in an assay described herein, are also within the scope of the invention.

In another aspect, a polypeptide consisting of or comprising a fragment of a connexin polypeptide having at least 10 contiguous amino acids of the connexin polypeptide is provided. In other embodiments, the fragment consists of at least 20 or 50 contiguous amino acids of the connexin polypeptide. In a specific embodiment, the fragments are not larger than 35, 100 or even 200 amino acids.

Fragments, derivatives or analogs of connexin polypeptide include but are not limited to those molecules comprising regions that are substantially similar to connexin polypeptide or fragments thereof (e.g., in various embodiments, at least 30%, 40%, 50%, 60%, 70%, 75%, 80%, 90%, or even 95% identity or similarity over an amino acid sequence of identical size), or when compared to an aligned sequence in which the alignment is done by a computer sequence comparison/alignment program known in the art, or whose coding nucleic acid is capable of hybridizing to a connexin nucleic acid, under high stringency, moderate stringency, or low stringency conditions well known to the skilled artisan.

The connexin polypeptide derivatives and analogs can be produced by various methods known in the art. The manipulations which result in their production can occur at the gene or protein level. For example, the cloned connexin nucleic acids can be modified by any of numerous strategies known in the art (see, e.g., Sambrook et al., (1989) supra), such as making conservative substitutions, deletions, insertions, and the like. The sequence can be cleaved at appropriate sites with restriction endonuclease(s), followed by further enzymatic modification if desired, isolated, and ligated in vitro. In the production of the connexin nucleic acids encoding a fragment, derivative or analog of a connexin polypeptide, the modified nucleic acid typically remains in the proper translational reading frame, so that the reading frame is not interrupted by translational stop signals or other signals which interfere with the synthesis of the connexin fragment, derivative or analog. The connexin nucleic acid can also be mutated in vitro or in vivo to create and/or destroy translation, initiation and/or termination sequences. The connexin encoding nucleic acid can also be mutated to create variations in coding regions and/or to form new restriction endonuclease sites or destroy preexisting ones and to facilitate further in vitro modification. Any technique for mutagenesis known in the art can be used, including but not limited to, chemical mutagenesis, in vitro site-directed mutagenesis (Hutchison et al., J. Biol. Chem. 253:6551-60 (1978)), the use of TAB® linkers (Pharmacia), and the like.

Manipulations of the connexin polypeptide sequence can also be made at the polypeptide level. Included within the scope of the invention are connexin polypeptide fragments, derivatives or analogs which are differentially modified during or after synthesis (e.g., in vivo or in vitro translation). Such modifications include conservative substitution, glycosylation, acetylation, phosphorylation, amidation, derivatization by known protecting/blocking groups, proteolytic cleavage, linkage to an antibody molecule or other cellular ligand, and the like). Any of numerous chemical modifications can be carried out by known techniques, including, but not limited to, specific chemical cleavage (e.g., by cyanogen bromide), enzymatic cleavage (e.g., by trypsin, chymotrypsin, papain, V8 protease, and the like); modification by, for example, NaBH₄ acetylation, formylation, oxidation and reduction, or metabolic synthesis in the presence of tunicamycin, and the like.

In addition, fragments, derivatives and analogs of connexin polypeptides can be chemically synthesized. For example, a peptide corresponding to a portion, or fragment, of a connexin polypeptide, which comprises a desired domain, or which mediates a desired activity in vitro, can be synthesized by use of chemical synthetic methods using, for example, an automated peptide synthesizer. Furthermore, if desired, non-classical amino acids or chemical amino acid analogs can be introduced as a substitution or addition into the connexin polypeptide sequence. Non-classical amino acids include but are not limited to the D-isomers of the common amino acids, α-amino isobutyric acid, 4-aminobutyric acid, 2-amino butyric acid, γ-amino butyric acid, ε-Ahx, 6-amino hexanoic acid, 2-amino isobutyric acid, 3-amino propionic acid, ornithine, norleucine, norvaline, hydroxyproline, sarcosine, citrulline, cysteic acid, t-butylglycine, t-butylalanine, phenylglycine, cyclohexylalanine, β-alanine, selenocysteine, fluoro-amino acids, designer amino acids such as β-methyl amino acids, C α-methyl amino acids, N α-methyl amino acids, and amino acid analogs in general. Furthermore, the amino acid can be D (dextrorotary) or L (levorotary).

In a specific embodiment, the connexin fragment or derivative is a chimeric, or fusion, protein comprising a connexin polypeptide or fragment thereof (typically consisting of at least a domain or motif of the connexin polypeptide, or at least 10 contiguous amino acids of the connexin polypeptide) joined at its amino- or carboxy-terminus via a peptide bond to an amino acid sequence of a different protein. In one embodiment, such a chimeric protein is produced by recombinant expression of a nucleic acid encoding the protein. The chimeric product can be made by ligating the appropriate nucleic acid sequence, encoding the desired amino acid sequences, to each other in the proper coding frame and expressing the chimeric product by methods commonly known in the art. Alternatively, the chimeric product can be made by protein synthetic techniques (e.g., by use of an automated peptide synthesizer).

Connexin polypeptide can be isolated and purified by standard methods including chromatography (e.g., ion exchange, affinity, sizing column chromatography, high pressure liquid chromatography), centrifugation, differential solubility, or by any other standard technique for the purification of proteins. The functional properties can be evaluated using any suitable assay as described herein or otherwise known to the skilled artisan. Alternatively, once a connexin polypeptide produced by a recombinant is identified, the amino acid sequence of the polypeptide can be deduced from the nucleotide sequence of the chimeric gene contained in the recombinant. As a result, the protein can be synthesized by standard chemical methods known in the art (see, e.g., Hunkapiller et al., Nature 310:105-11 (1984); Stewart and Young, Solid Phase Peptide Synthesis, 2nd Ed., Pierce Chemical Co., Rockford, Ill., (1984)).

In another alternate embodiment, native connexin polypeptides can be purified from natural sources by standard methods such as those described above (e.g., immunoaffinity purification). In a specific embodiment of the present invention, connexin polypeptides, whether produced by recombinant DNA techniques, by chemical synthetic methods or by purification of native polypeptides, include but are not limited to those containing as a primary amino acid sequence all or part of the amino acid sequence of human connexin polypeptide, as well as fragments, derivatives and analogs thereof.

Therapeutic Uses of Nucleic Acids Encoding Connexin Protein, Fragments, Derivatives, and Analogs.

The invention provides for treatment or prevention of various diseases and disorders by administration of a therapeutic compound (termed herein “Therapeutic”). Such “Therapeutics” include but are not limited to connexin proteins, derivatives, analogs and fragments thereof (e.g., as described herein above); nucleic acids encoding the connexin proteins, fragments, derivatives, and analogs (e.g. as described herein above); connexin anti-sense nucleic acids or other agents which act as agonists of connexin. Typically, the protein, fragment, polypeptide, derivative, or nucleic acid is administered in combination with a chemotherapeutic drug. The combination increasing the sensitivity of the target cells to the chemotherapeutic drug.

Disorders involving tumorigenesis or cell over-proliferation are treated or prevented by administration of a Therapeutic that promotes connexin function. Disorders in which cell proliferation is deficient or is desired are treated or prevented by administration of a Therapeutic that inhibits connexin function. See details in the subsections below.

Generally, it is preferred to administer a product of a species origin or species reactivity that is the same as that of the recipient. Thus, in a typical embodiment, a human connexin protein, derivative, or analog, or nucleic acid, is therapeutically or prophylactically administered to a human patient.

Chemotherapeutic Drugs.

There are five major classes of chemotherapeutic agents currently in use for the treatment of cancer. These include, natural products and their derivatives; anthracyclins; alkylating agents; antimetabolites; and hormonal agents. Chemotherapeutic agents are frequently referred to as antineoplastic agents.

The alkylating agents are believed act by alkylating and crosslinking guanine and possibly other vases in DNA, arresting cell division. Typical alkylating agents include nitrogen mustards, ethyleneimine compounds, alkyl sulfates, cisplatin, and various nitrosoureas.

Antimetabolites are typically reversible or irreversible enzyme inhibitors, or compounds that otherwise interfere with the replication, translation or transcription of nucleic acids.

Several synthetic nucleosides have been identified that exhibit anticancer activity. A well known nucleoside derivative with strong anticancer activity is 5-fluorouracil. 5-Fluorouracil has been used clinically in the treatment of malignant tumors, including for example, carcinomas, sarcomas, skin cancer, cancer of the digestive organs, and breast cancer.

The dosages required for clinical use in treating various cancers are well known. As are the typical routes of administration. A benefit of the present invention is that the combination of expression of a connexin and a neoplastic agent is that the effective dosage of the agent required can be lowered to below the usual dosage. This can reduce the possibility of increased resistance of the cancer cells to the drug.

When the anticancer agent is used in combination, the agent can be administered at the same time, prior to or subsequent with the connexin polypeptide, peptide, derivative or analog thereof, or a nucleic acid coding for the polypeptide. Further, combinations of antineoplastic agents can also be used.

Treatment and Prevention of Disorders Involving Over-Proliferation of Cells.

Diseases and disorders involving cell over-proliferation are treated or prevented by administration of a Therapeutic that promotes connexin function. Examples of such a Therapeutic include but are not limited to nucleic acids encoding connexin protein, derivatives, analogs or fragments thereof, under the control of a strong inducible promoter, particularly that are active in inhibiting cell proliferation (e.g., as demonstrated in in vitro assays or in animal models). Other Therapeutics that can be used, e.g., connexin, connexin peptides, peptide mimetics, or agents which increase the expression of connexin can be identified using in vitro assays or animal models, examples of which are described infra. In addition, a Therapeutic can include combinations of the above agents and molecules that promote connexin function combined with a chemotherapeutic agent or antineoplastic agent. The Therapeutic can also include an agent or molecule that inhibits the activity of MCP-1.

In specific embodiments, Therapeutics that promote connexin function and reduce the expression of bcl-2 and/or increase the effectiveness of chemotherapeutic drugs are administered therapeutically (including prophylactically): (1) in diseases or disorders involving a decreased (relative to normal or desired) level of connexin protein or function, for example, in patients where connexin protein is under-expressed, genetically defective, or biologically hypoactive; or (2) in diseases or disorders wherein in vitro (or in vivo) assays (see infra) indicate the utility of connexin agonist administration, for example, where bcl-2 is over-expressed. The decreased level in connexin protein or function can be readily detected, e.g., by obtaining a patient tissue sample (e.g. from biopsy tissue) and assaying it in vitro for RNA or protein levels, structure and/or activity of the expressed connexin RNA or protein. Many methods standard in the art can be thus employed, including but not limited to immunoassays to detect and/or visualize connexin protein (e.g., Western blot, immunoprecipitation followed by sodium dodecyl sulfate polyacrylamide gel electrophoresis, immunocytochemistry, and the like) and/or hybridization assays to detect connexin expression by detecting and/or visualizing connexin mRNA (e.g., Northern assays, dot blots, in situ hybridization, and the like).

Diseases and disorders involving cell over-proliferation that can be treated or prevented include but are not limited to malignancies, premalignant conditions (e.g., hyperplasia, metaplasia, dysplasia), benign tumors, hyperproliferative disorders, benign dysproliferative disorders, and the like. Malignancies and related disorders that can be treated or prevented by administration of a Therapeutic that promotes connexin function include but are not limited to carcinomas, adenocarcinomas, sarcomas, lymphomas, leukemia, and the like. In specific embodiments, malignancy or dysproliferative changes (such as metaplasia and dysplasias), or hyperproliferative disorders, are treated or prevented in the brain, breast, colon, prostate, lung, or skin. In other specific embodiments a carcinoma such as glioblastoma is treated or prevented.

The Therapeutics of the invention that agonize and promote connexin activity can also be administered to treat premalignant conditions and to prevent progression to a neoplastic or malignant state. Such prophylactic or therapeutic use is indicated in conditions known or suspected of preceding progression to neoplasia or cancer, in particular, where non-neoplastic cell growth consisting of hyperplasia, metaplasia, or most particularly, dysplasia has occurred (for review of such abnormal growth conditions, see Robbins and Angell, Basic Pathology, 2d Ed., W. B. Saunders Co., PA, pp. 68-79 (1972)). Hyperplasia is a form of controlled cell proliferation involving an increase in cell number in a tissue or organ, without significant alteration in structure or function. As but one example, endometrial hyperplasia often precedes endometrial cancer. Metaplasia is a form of controlled cell growth in which one type of adult or fully differentiated cell substitutes for another type of adult cell. Metaplasia can occur in epithelial or connective tissue cells. A typical metaplasia involves a somewhat disorderly metaplastic epithelium. Dysplasia is frequently a forerunner of cancer, and is found mainly in the epithelia; it is the most disorderly form of non-neoplastic cell growth, involving a loss in individual cell uniformity and in the architectural orientation of cells. Dysplastic cells often have abnormally large, deeply stained nuclei, and exhibit pleomorphism. Dysplasia characteristically occurs where there exists chronic irritation or inflammation, and is often found in the cervix, respiratory passages, oral cavity, and gall bladder.

Alternatively or in addition to the presence of abnormal cell growth characterized as hyperplasia, metaplasia, or dysplasia, the presence of one or more characteristics of a transformed phenotype, or of a malignant phenotype, displayed in vivo or displayed in vitro by a cell sample from a patient, can indicate the desirability of prophylactic/therapeutic administration of a Therapeutic that inhibits connexin function. As mentioned supra, such characteristics of a transformed phenotype include morphology changes, looser substratum attachment, loss of contact inhibition, loss of anchorage dependence, protease release, increased sugar transport, decreased serum requirement, expression of fetal antigens, and the like (see also Id., at pp. 84-90 for characteristics associated with a transformed or malignant phenotype).

In other embodiments, a patient which exhibits one or more of the following predisposing factors for malignancy is treated by administration of an effective amount of a Therapeutic: a chromosomal translocation associated with a malignancy (e.g., the Philadelphia chromosome for chronic myelogenous leukemia, t(14;18) for follicular lymphoma, and the like), familial polyposis or Gardner's syndrome (possible forerunners of colon cancer), benign monoclonal gammopathy (a possible forerunner of multiple myeloma), and a first degree kinship with persons having a cancer or precancerous disease showing a Mendelian (genetic) inheritance pattern (e.g., familial polyposis of the colon, Gardner's syndrome, hereditary exostosis, polyendocrine adenomatosis, medullary thyroid carcinoma with amyloid production and pheochromocytoma, Peutz-Jeghers syndrome, neurofibromatosis of Von Recklinghausen, retinoblastoma, carotid body tumor, cutaneous melanocarcinoma, intraocular melanocarcinoma, xeroderma pigmentosum, ataxia telangiectasia, Chediak-Higashi syndrome, albinism, Fanconi's aplastic anemia, and Bloom's syndrome; see Robbins and Angell, Basic Pathology, 2d Ed., W. B. Saunders Co., PA, pp. 112-113 (1976)) and the like.).

In another specific embodiment, a Therapeutic of the invention is administered to a human patient to prevent progression to brain, breast, colon, prostate, lung, or skin. In other specific embodiments, carcinoma, melanoma, or leukemia is treated or prevented.

Gene Therapy.

Gene therapy refers to therapy performed by the administration of a nucleic acid to a subject. In this embodiment of the invention, the nucleic acid mediates a therapeutic effect by increasing connexin transcription and translation.

Any of the methods for gene therapy available in the art can be used according to the present invention. Exemplary methods are described below.

For general reviews of the methods of gene therapy, see Goldspiel et al. Clinical Pharmacy 12:488-505 (1993); Wu and Wu, Biotherapy 3:87-95 (1991); Tolstoshev, Ann. Rev. Pharmacol. Toxicol. 32:573-596 (1993); Mulligan, Science 260:926-932 (1993); and Morgan and Anderson, Ann. Rev. Biochem. 62:191-217 (1993); TIBTECH 11:155-215 (1993)). Methods commonly known in the art of recombinant DNA technology which can be used are described in Ausubel et al. (eds.), Current Protocols in Molecular Biology, John Wiley & Sons, NY (1993); and Kriegler, Gene Transfer and Expression, A Laboratory Manual, Stockton Press, NY (1990).

In one embodiment, the Therapeutic comprises an connexin sense nucleic acid that is part of an expression vector that expresses a connexin protein or fragment or chimeric protein thereof in a suitable host. In particular, such a nucleic acid has a promoter operably linked to the connexin coding region, said promoter being inducible or constitutive, and, optionally, tissue-specific. In another particular embodiment, a nucleic acid molecule is used in which the connexin coding sequences and any other desired sequences are flanked by regions that promote homologous recombination at a desired site in the genome, thus providing for intrachromosomal expression of the connexin nucleic acid (Koller and Smithies, Proc. Natl. Acad. Sci. USA 86:8932-8935 (1989); Zijlstra et al., Nature 342:435-438 (1989)).

Delivery of the nucleic acid into a patient may be either direct, in which case the patient is directly exposed to the nucleic acid or nucleic acid-carrying vector, or indirect, in which case, cells are first transformed with the nucleic acid in vitro, then transplanted into the patient. These two approaches are known, respectively, as in vivo or ex vivo gene therapy.

In a specific embodiment, the nucleic acid is directly administered in vivo, where it is expressed to produce the encoded product. This can be accomplished by any of numerous methods known in the art, e.g., by constructing it as part of an appropriate nucleic acid expression vector and administering it so that it becomes intracellular, e.g. by infection using a defective or attenuated retroviral or other viral vector (see U.S. Pat. No. 4,980,286), or by direct injection of naked DNA, or by use of microparticle bombardment (e.g., a gene gun; Biolistic, Dupont), or coating with lipids or cell-surface receptors or transfecting agents, encapsulation in liposomes, microparticles, or microcapsules, or by administering it in linkage to a peptide which is known to enter the nucleus, by administering it in linkage to a ligand subject to receptor-mediated endocytosis (see e.g., Wu and Wu, J. Biol. Chem. 262:4429-4432 (1987)) (which can be used to target cell types specifically expressing the receptors), and the like. In another embodiment, a nucleic acid-ligand complex can be formed in which the ligand comprises a fusogenic viral peptide to disrupt endosomes, allowing the nucleic acid to avoid lysosomal degradation.

In yet another embodiment, the nucleic acid can be targeted in vivo for cell specific uptake and expression, by targeting a specific receptor (see, e.g., PCT Publications WO 92/06180; WO 92/22635; WO 92/20316; WO 93/14188, and WO 93/20221). Alternatively, the nucleic acid can be introduced intracellularly and incorporated within host cell DNA for expression, by homologous recombination (Koller and Smithies, Proc. Natl. Acad. Sci. USA 86:8932-8935 (1989); Zijlstra et al., Nature 342:435438 (1989)).

In a specific embodiment, a viral vector that contains the connexin nucleic acid is used. For example, a retroviral vector can be used (see Miller et al., Meth. Enzymol. 217:581-599 (1993)). These retroviral vectors have been modified to delete retroviral sequences that are not necessary for packaging of the viral genome and integration into host cell DNA. The connexin nucleic acid to be used in gene therapy is cloned into the vector, which facilitates delivery of the gene into a patient. More detail about retroviral vectors can be found in Boesen et al., Biotherapy 6:291-302 (1994), which describes the use of a retroviral vector to deliver the mdr1 gene to hematopoietic stem cells in order to make the stem cells more resistant to chemotherapy. Other references illustrating the use of retroviral vectors in gene therapy are: Clowes et al., J. Clin. Invest. 93:644-651 (1994); Kiem et al., Blood 83:1467-1473 (1994); Salmons and Gunzberg, Human Gene Therapy 4:129-141 (1993); and Grossman and Wilson, Curr. Opin. in Genetics and Devel. 3:110-114 (1993).

Adenoviruses are other viral vectors that can be used in gene therapy. Adenoviruses are especially attractive vehicles for delivering genes to respiratory epithelia. Adenoviruses naturally infect respiratory epithelia where they cause a mild disease. Other targets for adenovirus-based delivery systems are liver, the central nervous system, endothelial cells, and muscle. Adenoviruses have the advantage of being capable of infecting non-dividing cells (Kozarsky and Wilson, Curr. Op. Genet. Dev. 3:499-503 (1993) present a review of adenovirus-based gene therapy. Bout et al., (Hum. Gene Ther. 5:3-10 (1994)) demonstrated the use of adenovirus vectors to transfer genes to the respiratory epithelia of rhesus monkeys. Other instances of the use of adenoviruses in gene therapy can be found in Rosenfeld et al., Science 252:431-434 (1991); Rosenfeld et al., Cell 68:143-155 (1992); and Mastrangeli et al., J. Clin. Invest. 91:225-234 (1993)).

Adeno-associated virus (AAV) has also been proposed for use in gene therapy (Walsh et al., Proc. Soc. Exp. Biol. Med. 204:289-300 (1993). Another approach to gene therapy involves transferring a gene to cells in tissue culture by such methods as electroporation, lipofection, calcium phosphate mediated transfection, or viral infection. Usually, the method of transfer includes the transfer of a selectable marker to the cells. The cells are then placed under selection to isolate those cells that have taken up and are expressing the transferred gene. Those cells are then delivered to a patient.

In this embodiment, the nucleic acid is introduced into a cell prior to administration in vivo of the resulting recombinant cell. Such introduction can be carried out by any method known in the art, including but not limited to transfection, electroporation, microinjection, infection with a viral or bacteriophage vector containing the nucleic acid sequences, cell fusion, chromosome-mediated gene transfer, microcell-mediated gene transfer, spheroplast fusion, and the like. Numerous techniques are known in the art for the introduction of foreign genes into cells (see e.g., Loeffler and Behr, Meth. Enzymol. 217:599-618 (1993); Cohen et al., Meth. Enzymol. 217:618-644 (1993); Cline, Pharmac. Ther. 29:69-92 (1985)) and can be used in accordance with the present invention, provided that the necessary developmental and physiological functions of the recipient cells are not disrupted. The technique should provide for the stable transfer of the nucleic acid to the cell, so that the nucleic acid is expressible by the cell and preferably heritable and expressible by its cell progeny.

The resulting recombinant cells can be delivered to a patient by various methods known in the art. In a particular embodiment, epithelial cells are injected, e.g., subcutaneously. In another embodiment, recombinant skin cells may be applied as a skin graft onto the patient. Recombinant blood cells (e.g., hematopoietic stem or progenitor cells) are preferably administered intravenously. The amount of cells envisioned for use depends on the desired effect, patient state, and the like, and can be determined by one skilled in the art.

Cells into which a nucleic acid can be introduced for purposes of gene therapy encompass any desired, available cell type, and include but are not limited to epithelial cells, endothelial cells, keratinocytes, fibroblasts, muscle cells, hepatocytes; blood cells such as T lymphocytes, B lymphocytes, monocytes, macrophages, neutrophils, eosinophils, megakaryocytes, granulocytes; various stem or progenitor cells, in particular hematopoietic stem or progenitor cells, e.g., as obtained from bone marrow, umbilical cord blood, peripheral blood, fetal liver, and the like. In a typical embodiment, the cell used for gene therapy is autologous to the patient.

In an embodiment in which recombinant cells are used in gene therapy, a connexin nucleic acid is introduced into the cells such that it is expressible by the cells or their progeny, and the recombinant cells are then administered in vivo for therapeutic effect. In a specific embodiment, stem or progenitor cells are used. Any stem and/or progenitor cells which can be isolated and maintained in vitro can potentially be used in accordance with this embodiment of the present invention. Such stem cells include but are not limited to hematopoietic stem cells (HSC), stem cells of epithelial tissues such as the skin and the lining of the gut, embryonic heart muscle cells, liver stem cells (PCT Publication WO 94/08598), and neural stem cells (Stemple and Anderson, Cell 71:973-985 (1992)).

Epithelial stem cells (ESCs) or keratinocytes can be obtained from tissues such as the skin and the lining of the gut by known procedures (Rheinwald, Meth. Cell Bio. 21A:229 (1980)). In stratified epithelial tissue such as the skin, renewal occurs by mitosis of stem cells within the germinal layer, the layer closest to the basal lamina. Stem cells within the lining of the gut provide for a rapid renewal rate of this tissue. ESCs or keratinocytes obtained from the skin or lining of the gut of a patient or donor can be grown in tissue culture (Rheinwald, Meth. Cell Bio. 21A:229 (1980); Pittelkow and Scott, Mayo Clinic Proc. 61:771 (1986)). If the ESCs are provided by a donor, a method for suppression of host versus graft reactivity (e.g., irradiation, drug or antibody administration to promote moderate immunosuppression) can also be used.

With respect to hematopoietic stem cells (HSC), any technique which provides for the isolation, propagation, and maintenance in vitro of HSC can be used in this embodiment of the invention. Techniques by which this may be accomplished include (a) the isolation and establishment of HSC cultures from bone marrow cells isolated from the future host, or a donor, or (b) the use of previously established long-term HSC cultures, which may be allogeneic or xenogeneic. Non-autologous HSC are used preferably in conjunction with a method of suppressing transplantation immune reactions of the future host/patient. In a particular embodiment of the present invention, human bone marrow cells can be obtained from the posterior iliac crest by needle aspiration (see, e.g., Kodo et al., J. Clin. Invest. 73:1377-1384 (1984)). In a preferred embodiment of the present invention, the HSCs can be made highly enriched or in substantially pure form. This enrichment can be accomplished before, during, or after long-term culturing, and can be done by any techniques known in the art. Long-term cultures of bone marrow cells can be established and maintained by using, for example, modified Dexter cell culture techniques (Dexter et al., J. Cell Physiol. 91:335 (1977)) or Witlock-Witte culture techniques (Witlock and Witte, Proc. Natl. Acad. Sci. USA 79:3608-3612 (1982)).

In a specific embodiment, the nucleic acid to be introduced for purposes of gene therapy comprises an inducible promoter operably linked to the coding region, such that expression of the nucleic acid is controllable by controlling the presence or absence of the appropriate inducer of transcription. Additional methods that can be adapted for use to deliver a nucleic acid encoding a connexin protein or functional derivative thereof are described herein.

Demonstration of Therapeutic or Prophylactic Utility.

The Therapeutics of the invention are preferably tested in vitro, and then in vivo for the desired therapeutic or prophylactic activity, prior to use in humans. For example, in vitro assays which can be used to determine whether administration of a specific therapeutic is indicated, include in vitro cell culture assays in which a patient tissue sample is grown in culture, and exposed to or otherwise administered a therapeutic, and the effect of the Therapeutic upon the tissue sample is observed. Typically, the connexin, polypeptide, fragment, derivative or analog, or nucleic acid sequence is combined with a chemotherapeutic agent and contacted with the test cells.

In one embodiment, where the patient has a malignancy, a sample of cells from such malignancy is plated out or grown in culture, and the cells are then exposed to a Therapeutic. A Therapeutic which inhibits survival or growth of the malignant cells is selected for therapeutic use in vivo. Many assays standard in the art can be used to assess such survival and/or growth; for example, cell proliferation can be assayed by measuring ³H-thymidine incorporation, by direct cell count, by detecting changes in transcriptional activity of known genes such as proto-oncogenes (e.g., fos, myc) or cell cycle markers; cell viability can be assessed by trypan blue staining, differentiation can be assessed visually based on changes in morphology, and the like. In addition, the cells can be assayed for a decrease in bcl-2 expression. The assay can determine the amount of bcl-2 protein expressed or can quantitate the amount of mRNA produced by the cell using standard methods well known to the skilled artisan. Also, effectiveness of the compositions of the present invention can be tested by contacting the cells with various concentrations of chemotherapeutic drug and connexin to determine whether there is an increase in sensitivity to the chemotherapeutic drug.

In another embodiment, a Therapeutic is indicated for use which exhibits the desired effect, inhibition or promotion of cell growth, upon a patient cell sample from tissue having or suspected of having a hyperproliferative disorder. Such hyperproliferative disorders include but are not limited to those described above. In various specific embodiments, in vitro assays can be carried out with representative cells of cell types involved in a patient's disorder, to determine if a Therapeutic has a desired effect upon such cell types.

In another embodiment, cells of a patient tissue sample suspected of being pre-neoplastic are similarly plated out or grown in vitro, and exposed to a Therapeutic.

The Therapeutic which results in a cell phenotype that is more normal (i.e., less representative of a pre-neoplastic state, neoplastic state, malignant state, or transformed phenotype) is selected for therapeutic use. Many assays standard in the art can be used to assess whether a pre-neoplastic state, neoplastic state, or a transformed or malignant phenotype, is present. For example, characteristics associated with a transformed phenotype (a set of in vitro characteristics associated with a tumorigenic ability in vivo) include a more rounded cell morphology, looser substratum attachment, loss of contact inhibition, loss of anchorage dependence, release of proteases such as plasminogen activator, increased sugar transport, decreased serum requirement, expression of fetal antigens, and the like. (see Luria et al., General Virology, 3d Ed., John Wiley & Sons, New York pp. 436-446 (1978)).

In other specific embodiments, the in vitro assays described supra can be carried out using a cell line, rather than a cell sample derived from the specific patient to be treated, in which the cell line is derived from or displays characteristic(s) associated with the malignant, neoplastic or pre-neoplastic disorder desired to be treated or prevented, or is derived from the cell type upon which an effect is desired, according to the present invention.

Compounds for use in therapy can be tested in suitable animal model systems prior to testing in humans, including but not limited to rats, mice, chicken, cows, monkeys, rabbits, and the like. For in vivo testing, prior to administration to humans, any animal model system known in the art may be used.

Therapeutic/Prophylactic Administration and Compositions.

The invention provides methods of treatment (and prophylaxis) by administration to a subject of an effective amount of a Therapeutic of the invention. In a particular aspect, the Therapeutic is substantially purified. The subject is preferably an animal, including but not limited to animals such as cows, pigs, horses, chickens, cats, dogs, and the like, and is typically a mammal, and preferably human. In a specific embodiment, a non-human mammal is the subject.

Formulations and methods of administration that can be employed when the Therapeutic comprises a nucleic acid are described above; additional appropriate formulations and routes of administration can be selected from among those described herein below. In a particular embodiment, the connexin protein, fragment, derivative or analog thereof, or a nucleic acid sequence encoding the connexin protein, fragment, derivative or analog thereof, is administered in combination with a chemotherapeutic drug. In another embodiment, the formulation can also include an antagonist of MCP-1 activity. The connexin protein or nucleic acid can be administered at the same time as the chemotherapeutic drug or antagonist of MCP-1 activity, but is usually administered separately.

Various delivery systems are known and can be used to administer a therapeutic of the invention, e.g., encapsulation in liposomes, microparticles, microcapsules, recombinant cells capable of expressing the Therapeutic, receptor-mediated endocytosis (see, e.g., Wu and Wu, J. Biol. Chem. 262:4429-4432 (1987)), construction of a Therapeutic nucleic acid as part of a retroviral or other vector, and the like. Methods of introduction include but are not limited to intradermal, intramuscular, intraperitoneal, intravenous, subcutaneous, intranasal, epidural, and oral routes. The compounds can be administered by any convenient route, for example, by infusion or bolus injection, by absorption through epithelial or mucocutaneous linings (e.g., oral mucosa, rectal and intestinal mucosa, and the like) and may be administered together with other biologically active agents. Administration can be systemic or local. In addition, it may be desirable to introduce the pharmaceutical compositions of the invention into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent.

In a specific embodiment, it may be desirable to administer the pharmaceutical compositions of the invention locally to the area in need of treatment; this may be achieved by, for example, and not by way of limitation, local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as silastic membranes, or fibers. In one embodiment, administration can be by direct injection at the site (or former site) of a malignant tumor or neoplastic or preneoplastic tissue.

In another embodiment, the Therapeutic can be delivered in a vesicle, in particular a liposome (see Langer, Science 249:1527-1533 (1990); Treat et al., in Liposomes in the Therapy of Infectious Disease and Cancer, Lopez-Berestein and Fidler (eds.), Liss, NY, pp. 353-365 (1989); Lopez-Berestein, ibid., pp. 317-327; see generally ibid.)

In yet another embodiment, the therapeutic can be delivered in a controlled release system. In one embodiment, a pump can be used (see Langer, supra; Sefton, C R C Crit. Ref. Biomed. Eng. 14:201 (1987); Buchwald et al., Surgery 88:507 (1980); Saudek et al., N. Engl. J. Med. 321:574 (1989)). In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, NY (1984); Ranger and Peppas, J. Macromol. Sci. Rev. Macromol. Chem. 23:61 (1983); see also Levy et al., Science 228:190 (1985); During et al., Ann. Neurol. 25:351 (1989); Howard et al., J. Neurosurg. 71:105 (1989)). In yet another embodiment, a controlled release system can be placed in proximity of the therapeutic target, i.e., the brain, thus requiring only a fraction of the systemic dose (see, e.g., Goodson, in Medical Applications of Controlled Release, supra Vol. 2, pp. 115-138 (1984)). Other controlled release systems are discussed in the review by Langer (Science 249:1527-1533 (1990)).

In a specific embodiment where the therapeutic is a nucleic acid encoding a protein therapeutic, the nucleic acid can be administered in vivo to promote expression of its encoded protein, by constructing it as part of an appropriate nucleic acid expression vector and administering it so that it becomes intracellular, e.g., by use of a retroviral vector (see, for example, U.S. Pat. Nos. 4,980,286; 5,580,766; 5,741,486; 5,886,166; 6,156,303; 6,171,855; 6,180,613; and the like), or by direct injection, or by use of microparticle bombardment (e.g., a gene gun; BIOLISTIC, Dupont), or coating with lipids or cell-surface receptors or transfecting agents, or by administering it in linkage to a homoeobox-like peptide which is known to enter the nucleus (see e.g. Joliot et al., Proc. Natl. Acad. Sci. USA 88:1864-1868 (1991)), and the like. Alternatively, a nucleic acid therapeutic can be introduced intracellularly and incorporated within host cell DNA for expression, by homologous recombination.

The present invention also provides pharmaceutical compositions. Such compositions comprise a therapeutically effective amount of a therapeutic, and a pharmaceutically acceptable carrier. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, and the like. Examples of suitable pharmaceutical carriers are described in Remington's Pharmaceutical Sciences by E. W. Martin. Such compositions will contain a therapeutically effective amount of the therapeutic, preferably in purified form, together with a suitable amount of carrier so as to provide the form for proper administration to the patient. The formulation should suit the mode of administration.

In a typical embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition can also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The Therapeutics of the invention can be formulated as neutral or salt forms. Pharmaceutically acceptable salts include those formed with free amino groups such as those derived from hydrochloric, phosphoric, acetic, oxalic, tartaric acids, and the like, and those formed with free carboxyl groups such as those derived from sodium, potassium, ammonium, calcium, ferric hydroxides, isopropylamine, triethylamine, 2-ethylamino ethanol, histidine, procaine, and the like.

The amount of the therapeutic of the invention which will be effective in the treatment of a particular disorder or condition will depend on the nature of the disorder or condition, and can be determined by standard clinical techniques. In addition, in vitro assays may optionally be employed to help identify optimal dosage ranges. The precise dose to be employed in the formulation will also depend on the route of administration, and the seriousness of the disease or disorder, and should be decided according to the judgment of the practitioner and each patient's circumstances. However, suitable dosage ranges for intravenous administration are generally about 20-500 micrograms of active compound per kilogram body weight. Suitable dosage ranges for intranasal administration are generally about 0.01 pg/kg body weight to 1 mg/kg body weight. Effective doses may be extrapolated from dose-response curves derived from in vitro or animal model test systems.

Suppositories generally contain active ingredient in the range of 0.5% to 10% by weight; oral formulations preferably contain 10% to 95% active ingredient.

The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

Detection of Expression of Connexin Protein and Selected Genes.

After a given cell is transduced with a nucleic acid construct that encodes a connexin protein and optionally a drug sensitivity gene, it is important to detect which cells and cell lines express connexin protein and to assess the level of expression of connexin protein or a chemotherapeutic drug. This requires the detection of nucleic acids that encode a connexin protein or bcl-2, and also the detection of the protein gene products.

Nucleic acids and proteins are detected and quantified herein by any of a number of means well known to those of skill in the art. These include analytic biochemical methods such as spectrophotometry, radiography, electrophoresis, capillary electrophoresis, high performance liquid chromatography (HPLC), thin layer chromatography (TLC), hyperdiffusion chromatography, and the like, and various immunological methods such as fluid or gel precipitin reactions, immunodiffusion (single or double), immunoelectrophoresis, radioimmunoassays (RIAs), enzyme-linked immunosorbent assays (ELISAs), immunofluorescence assays, tissue array, and the like. The detection of nucleic acids proceeds by well known methods such as Southern analysis, Northern analysis, dot blot analysis, cDNA arrays, gel electrophoresis, PCR, radiolabeling, scintillation counting, and affinity chromatography.

Detection of Nucleic Acids Encoding Connexin Protein.

A variety of methods of specific DNA and RNA measurements and nucleic acid hybridization techniques known to those of skill in the art are useful for detecting and quantifying the presence and expression of connexin protein or pro-drug activating molecules. For example, one method for evaluating the presence of connexin protein DNA in a sample involves a Southern transfer. Southern et al., J. Mol. Biol. 98:503 (1975). Briefly, the digested genomic DNA is run on agarose slab gels in buffer and transferred to membranes. Hybridization is carried out using probes that recognize a connexin protein sequence.

Similarly, a Northern transfer can be used for the detection of connexin protein mRNA in samples of RNA from engineered cells that express the connexin protein gene. In brief, the mRNA is isolated from a given cell sample using an acid guanidinium-phenol-chloroform extraction method. The mRNA is then electrophoresed to separate the mRNA species and the mRNA is transferred from the gel to a nitrocellulose membrane. As with the Southern blots, labeled probes are used to identify the presence or absence of a connexin protein transcript.

The selection of a nucleic acid hybridization format is not critical. A variety of nucleic acid hybridization formats are known to those skilled in the art. For example, common formats include sandwich assays and competition or displacement assays. Hybridization techniques are generally described in Nucleic Acid Hybridization, A Practical Approach, ed. Hames and Higgins, IRL Press, (1985).

The sensitivity of the hybridization assays may be enhanced through use of a nucleic acid amplification system which multiplies the target nucleic acid being detected. In vitro amplification techniques suitable for amplifying sequences for use as molecular probes or for generating nucleic acid fragments for subsequent subcloning are known. Examples of techniques sufficient to direct persons of skill through such in vitro amplification methods, including the polymerase chain reaction (PCR) the ligase chain reaction (LCR), Q,β-replicase amplification and other RNA polymerase mediated techniques (e.g., NASBA) are found in Berger, Sambrook, and Ausubel, as well as Mullis et al., U.S. Pat. No. 4,683,202 (1987); PCR Protocols A Guide to Methods and Applications (Innis et al. eds), Academic Press Inc., San Diego, Calif. (1990) (Innis); Arnheim & Levinson (Oct. 1, 1990), Chem. Engineer. News, 36-47; Kwoh et al., J. NIH Res, 3:81-94 (1991); Kwoh et al., Proc. Natl. Acad. Sci. USA 86:1173 (1989); Guatelli et al., Proc. Natl. Acad. Sci. USA 87:1874 (1990); Lomell et al., J. Clin. Chem. 35:1826 (1989); Landegren et al., Science 241:1077-1080 (1988); van Brunt, Biotechnology 8:291-294 (1990); Wu and Wallace, Gene 4:560 (1989); Barringer et al., Gene 89:117 (1990), and Sooknanan and Malek, Biotechnology 13:563-564 (1995). Improved methods of cloning in vitro amplified nucleic acids are described in Wallace et al., U.S. Pat. No. 5,426,039. Other methods recently described in the art are the nucleic acid sequence based amplification (NASBA, Cangene, Mississauga, Ontario) and Q Beta Replicase systems. These systems can be used to directly identify mutants where the PCR or LCR primers are designed to be extended or ligated only when a select sequence is present. Alternatively, the select sequences can be generally amplified using, for example, nonspecific PCR primers and the amplified target region later probed for a specific sequence indicative of a mutation.

Oligonucleotides for use in in vitro amplification methods, for use as gene probes, or as inhibitor components are typically synthesized chemically according to the solid phase phosphoramidite triester method described by Beaucage and Caruthers, Tetrahedron Letts. 22:1859-1862 (1981), e.g., using an automated synthesizer, as described in Needham-VanDevanter et al., Nucleic Acids Res. 12:6159-6168 (1984). Purification of oligonucleotides, where necessary, is typically performed by either native acrylamide gel electrophoresis or by anion-exchange HPLC as described in Pearson and Regnier, J. Chrom. 255:137-149 (1983). The sequence of the synthetic oligonucleotides can be verified using the chemical degradation method of Maxam and Gilbert, Meth. Enzymol. 65:499-560 (1980).

An alternative means for determining the level of expression of connexin mRNA is in situ hybridization. In situ hybridization assays are well known and are generally described in Angerer et al., Meth. Enzymol. 152:649-660 (1987). In an in situ hybridization assay cells are fixed to a solid support, typically a glass slide. If DNA is to be probed, the cells are denatured with heat or alkali. The cells are then contacted with a hybridization solution at a moderate temperature to permit annealing of connexin protein-specific probes that are labeled. The probes are preferably labeled with radioisotopes or fluorescent reporters.

The presence of a connexin polypeptide (including peptide or enzymatic digestion product) in a sample may be detected and quantified using Western blot analysis. The technique generally comprises separating sample products by gel electrophoresis on the basis of molecular weight, transferring the separated proteins to a suitable solid support, (such as a nitrocellulose filter, a nylon filter, or derivatized nylon filter), and incubating the sample with labeling antibodies that specifically bind to the analyte protein. The labeling antibodies specifically bind to analyte on the solid support. These antibodies are directly labeled, or alternatively are subsequently detected using labeling agents such as antibodies (e.g., labeled sheep anti-mouse antibodies where the antibody to an analyte is a murine antibody) that specifically bind to the labeling antibody.

Diagnosis and Screening.

Connexin polypeptides and connexin nucleic acids, and fragments, derivatives, and analogs thereof, also have utility in diagnostics. Such molecules can be used in assays, such as to detect, prognose, diagnose, or monitor neoplastic disorders, or to monitor the treatment thereof. In particular, methods, such as an immunoassay, can be carried out by steps comprising contacting a sample derived from a patient with an anti-connexin antibody under conditions conducive to immunospecific binding, and detecting or measuring the amount of any immunospecific binding by the antibody. In a particular aspect, binding of antibody to connexin polypeptide, in tissue sections, can be used to detect aberrant connexin localization or aberrant (e.g., low, absent or elevated) levels of connexin polypeptide. In a specific embodiment, antibody to connexin polypeptide can be used to assay a patient tissue or serum sample for the presence of connexin, where an aberrant level of connexin is an indication of a disease. By “aberrant levels” is meant increased or decreased levels relative to that present, or a standard level representing that present, in an analogous sample from a portion of the body or from a subject not having the disease.

The immunoassays which can be used include, but are not limited to, competitive and non-competitive assay systems using techniques such as Western blot, radioimmunoassay, ELISA (enzyme linked immunosorbent assay), “sandwich” immunoassay, immunoprecipitation assay, precipitin reaction, gel diffusion precipitin reaction, immunodiffusion assay, agglutination assay, complement-fixation assay, immunoradiometric assay, fluorescent immunoassay, protein A immunoassay, tissue arrays, and the like.

Connexin genes and related nucleic acid sequences and subsequences, including complementary sequences, can also be used in hybridization assays. Connexin nucleic acid sequences (e.g., connexin 43, Fishman et al., J. Cell Biol. 111:589-598 (1990), incorporated herein by reference), or fragments thereof comprising about at least 8 nucleotides, can be used as hybridization probes. Hybridization assays can be used to detect, prognose, diagnose, or monitor disease (including conditions and disorders) associated with aberrant changes in connexin expression and/or activity, as described supra. In particular, a hybridization assay is carried out by a method comprising contacting a sample containing polynucleotides with a nucleic acid probe capable of hybridizing to connexin DNA or RNA, under conditions such that hybridization can occur, and detecting or measuring any resulting hybridization. In particular, the level of connexin produced can be compared to the expression level of bcl-2. A high connexin expression level coupled with lower bcl-2 expression is considered a better prognosis than a high bcl-2 expression level and low level of connexin expression.

In specific embodiments, diseases involving hyper-proliferation of cells can be diagnosed, or their suspected presence can be screened for, or a predisposition to develop such diseases can be identified by detecting decreased or increased levels of connexin polypeptide, connexin RNA, or connexin functional activity. Additionally, hyper-proliferation can be diagnosed by detecting mutations in connexin RNA or DNA or connexin polypeptide (e.g., translocations in connexin nucleic acids, truncations in the connexin gene or connexin polypeptide, changes in nucleotide or amino acid sequence relative to wild-type connexin, or connexin, respectively) that cause decreased or increased expression or activity of connexin polypeptide.

By way of example, levels of connexin polypeptide in a biopsy can be detected by immunoassay; levels of connexin RNA can be detected by hybridization assays (e.g., Northern blot or dot blot). Translocations and point mutations in connexin nucleic acids can be detected by Southern blot, RFLP analysis, PCR using primers that typically generate a fragment spanning at least most of the connexin gene, sequencing of the connexin genomic DNA or cDNA obtained from the sample, and the like.

In one embodiment, levels of connexin mRNA or connexin polypeptide in a sample of a tissue isolated from a patient are detected or measured, in which increased levels indicate that the subject has, or has a predisposition to developing, a malignancy or hyper-proliferative disease of that tissue, and in which the increased levels are relative to the levels present in an analogous sample from a portion of the body or from a subject not having the malignancy or other hyper-proliferative disease, as the case may be.

In another specific embodiment, diseases involving a deficiency in cell proliferation or in which cell proliferation is desirable for treatment, are diagnosed, or their suspected presence can be screened for, or a predisposition to develop such diseases can be detected, by detecting decreased levels of connexin polypeptide or connexin mRNA. Additionally, a deficiency in cell proliferation can be diagnosed by detecting connexin functional activity, or by detecting mutations in connexin RNA or DNA or connexin polypeptide (for example, translocations in connexin nucleic acids, truncations in the gene or polypeptide, changes in nucleotide or amino acid sequence relative to wild-type connexin gene or connexin polypeptide) that cause decreased expression or activity of connexin. By way of example, levels of connexin polypeptide, levels of connexin mRNA, connexin binding activity, and the presence of translocations or point mutations in the connexin gene can be determined as described above.

In a specific embodiment, levels of connexin mRNA or connexin polypeptide in a patient sample are detected or measured, in which decreased levels indicate that the subject has, or has a predisposition to developing, a hypo-proliferative disorder, in which the decreased levels are relative to the levels present in an analogous sample from a portion of the body or from a subject not having the hypo-proliferative disorder, as the case may be.

Kits for diagnostic use are also provided that comprise, in one or more containers, an anti-connexin antibody and, optionally, a labeled binding partner to the antibody. Alternatively, the anti-connexin antibody can be labeled with a detectable marker (e.g., a chemiluminescent, enzymatic, fluorescent, a radioactive moiety, and the like). A kit is also provided that comprises, in one or more containers, a nucleic acid probe capable of hybridizing to connexin mRNA.

In a specific embodiment, a kit can comprise in one or more containers a pair of primers (e.g., each in the size range of 6-30 nucleotides or more) that are capable of priming amplification (e.g., by polymerase chain reaction (see, e.g., Innis et al., PCR Protocols, Academic Press, Inc., San Diego, Calif. (1989)), ligase chain reaction (see, e.g., EP 320 308), use of Qβ replicase, cyclic 5′ probe reaction, or other methods known in the art) under appropriate reaction conditions such that at least a portion of a connexin nucleic acid is amplified. A kit can optionally further comprise in a container a predetermined amount of a purified connexin polypeptide or connexin nucleic acid, for example, for use as a standard or control.

In another embodiment the kit can comprise antibody conjugated, or labeled, with an oligonucleotide (DNA or RNA) to serve as an amplification system such as in PCR ELISA (see e.g., Landgraf et al., Anal. Biochem. 198:86-91 (1991)) and immuno-RCA (rolling circle amplification; (see, e.g., Schweilze et al., Proc. Natl. Acad. Sci. USA 97:10113-10119 (2000); Hatch et al., Genet. Anal. 15:35-40 (1999)) assays.

Screening for New Chemotherapy Compounds or Agents.

Connexin nucleic acids, connexin polypeptide, and fragments, derivatives and analogs thereof, also have uses in screening assays to detect candidate compounds that enhance chemotherapy induced apoptosis in target cells. The compounds or agent can be identified by in vitro and/or in vivo assays. Such assays can be used to identify agents that are therapeutically effective, such as anti-proliferative agents, or as lead compounds for drug development. The invention thus provides assays to detect candidate compounds and agents that specifically affect the activity or expression of connexin nucleic acids, connexin polypeptides, or fragments, derivatives or analogs thereof in enhancing chemotherapy induced apoptosis.

In a typical in vivo assay, recombinant cells expressing connexin nucleic acids can be used to screen candidate compounds for those that affect connexin and bcl-2 expression. Effects on connexin and/or bcl-2 expression can include transcription of mRNA, translation of the mRNA, synthesis of connexin and/or bcl-2 polypeptides, effects on connexin and/or bcl-2 polypeptide function (e.g., rRNA synthesis) and on connexin and/or bcl-2 polypeptide stability or localization. Such effects on connexin and/or bcl-2 expression can be identified as physiological changes, such as, for example, changes in cell growth rate, division, viability or morphological changes associated with apoptotic cells. In one embodiment, candidate compounds are administered to recombinant cells expressing connexin polypeptide to identify those compounds that produce a physiological change. In another embodiment, the method comprises administering a candidate compound to a first cell that expresses a first connexin polypeptide; administering the candidate compound to a second cell that expresses a second connexin polypeptide; and determining whether the candidate compound modulates the activity of the first connexin polypeptide but not the activity of the second connexin polypeptide. For example, the first connexin polypeptide can be yeast connexin polypeptide and the second can be human connexin polypeptide. Alternatively, the first connexin polypeptide can be a mutant, and the second connexin polypeptide can be wild-type.

Candidate compounds can also be identified by in vitro screens. For example, recombinant cells expressing connexin nucleic acids can be used to recombinantly produce connexin polypeptide for in vitro assays to identify candidate compounds that enhance the sensitivity of the cells to a chemotherapeutic drug. Candidate compounds (such as connexin polypeptides, peptide mimetics, or small molecules) are contacted with the connexin polypeptide (or fragment, derivative or analog thereof) under conditions conducive to cell proliferation, and then candidate compounds which demonstrate increased sensitivity to a chemotherapeutic drug are identified. Similar methods can be used to screen for candidate compounds that bind to nucleic acids encoding connexin, or a fragment, derivative or analog thereof. Methods that can be used to carry out the foregoing are commonly known in the art, and include diversity libraries, such as random or combinatorial peptide or non-peptide libraries that can be screened for candidate compounds that enhance the sensitivity of a cell population to a chemotherapeutic drug. Many libraries are known in the art that can be used, for example, include chemically synthesized libraries, recombinant phage display libraries, and in vitro translation-based libraries.

Examples of chemically synthesized libraries are described in Fodor et al., (Science 251:767-73 (1991)), Houghten et al. (Nature 354:84-86 (1991)), Lam et al., (Nature 354:82-84 (1991)), Medynski (Bio/Technology 12:709-10 (1994)), Gallop et al., (J. Med. Chem. 37:1233-51 (1994)), Ohlmeyer et al., (Proc. Nat. Acad. Sci. USA 90:10922-26 (1993)), Erb et al., (Proc. Natl. Acad. Sci. USA 21:11422-26 (1994)), Houghten et al., (Biotechniques 13:412-21 (1992)), Jayawickreme et al., (Proc. Natl. Acad. Sci. USA 91:1614-18 (1994)), Salmon et al., (Proc. Nat. Acad. Sci. USA 90:11708-12 (1993)), International Patent Publication WO 93/20242, and Brenner and Lerner (Proc. Natl. Acad. Sci. USA 89:5381-83 (1992)).

Examples of phage display libraries are described in Scott and Smith (Science 249:386-90 (1990)), Devlin et al., (Science 249:404-06 (1990)), Christian et al., (J. Mol. Biol. 227:711-18 (1992)), Lenstra (J. Immunol. Meth. 152:149-57 (1992)), Kay et al., (Gene 128:59-65 (1993)), and International Patent Publication WO 94/18318.

In vitro translation-based libraries include, but are not limited to, those described in International Patent Publication WO 91/05058, and Mattheakis et al., (Proc. Nat. Acad. Sci. USA 21:9022-26 (1994)). By way of examples of non-peptide libraries, a benzodiazepine library (see, e.g., Bunin et al., Proc. Nat. Acad. Sci. USA 21:4708-12 (1994)) can be adapted for use. Peptide libraries (see, e.g., Simon et al., Proc. Natl. Acad. Sci. USA 89:9367-71 (1992)) can also be used. Another example of a library that can be used, in which the amide functionalities in peptides have been permethylated to generate a chemically transformed combinatorial library, is described by Ostresh et al., (Proc. Natl. Acad. Sci. USA 91:11138-42 (1994)).

Screening the libraries can be accomplished by any of a variety of commonly known methods. See, for example, the following references, which disclose screening of peptide libraries: Parmley and Smith (Adv. Exp. Med. Biol. 251:215-18 (1989)); Scott and Smith ((1990) supra); Fowlkes et al., (BioTechniques 13:422-28 (1992)); Oldenburg et al., (Proc. Natl. Acad. Sci. USA 89:5393-97 (1992)); Yu et al., (Cell 76:933-45 (1994)); Staudt et al., (Science 241:577-80 (1988)); Bock et al., Nature 355:564-66 (1992)); Tuerk et al., (Proc. Natl. Acad. Sci. USA 89:6988-92 (1992)); Ellington et al., (Nature 355:850-52 (1992)); U.S. Pat. Nos. 5,096,815, 5,223,409, and 5,198,346; Rebar and Pabo (Science 263:671-73 (1994)); and International Patent Publication WO 94/18318.

In a specific embodiment, screening can be carried out by contacting the library members with a target cell population and harvesting those library members that demonstrate an effect on the proliferation of the cell population when exposed to a chemotherapeutic drug.

Selection of Patients.

The patients to be treated by the methods of the invention are cancer patients. The claimed methods are effective against a range of different cancer types. Typically, thee cancer is a tumor-forming cancer. For example, many solid tumors are amenable to treatment using the claimed invention. These tumors include but are not limited to tumors of neuroectodermal derivation (e.g., glioma), carcinomas (e.g., colon cancer, ovarian cancer), and tumors of mesodermal origin (e.g., sarcomas).

In order to assess how well the methods of the invention may be expected to work, the clinician can pre-test the efficacy of the treatment of a particular tumor type either in vitro or in vivo.

For in vitro tests, cells derived from the tumor are grown in tissue culture. The growth or proliferation inhibiting effect can be assessed using a number of commonly used assays, such as cell counts, or radioactive thymidine incorporation, or a methylcellulose assay (Lunardi-Iskandar et al., Clin. Exp. Immunol. 60:285-293 (1985)).

Administration of nucleic acid sequences encoding connexin protein to a cancer patient can be achieved in various ways known to skilled practitioners. The nucleic acid can be injected intratumorly: the tumor, the placement of the needle and release of the contents of the syringe may be visualized either by direct observation (for easily accessible tumors such as surface tumors or tumors easily exposed by surgical techniques), by endoscopic visualization, or by electromagnetic imaging techniques such as ultrasound, magnetic resonance imaging (MRI), CT scans. The nucleic acid can also be administered via injection into the bloodstream using a cannula or catheter; the vein or artery is selected to maximize delivery of cells to the tumor or affected tissue. The cells can be injected into cerebro-spinal fluid (i.e., into intracisternal, intraventricular, intrathecal or subarachnoid compartments). In cystic or vesicular tumors or tissues, the cells may be delivered intracystically or intravesicularly.

It is contemplated that the nucleic acid will be administered under the guidance of a physician. The concentration of nucleic acid to be administered at a given time and to a given patient will vary. Generally, the amount of nucleic acid to be administered is the amount necessary to reduce bcl-2 expression and subsequently, cancer cell growth and/or to destroy cancer cells and/or preferably to eradicate the cancer. More than one administration may be necessary. As with any medical treatment, the supervising physician will monitor the progress of the treatment, and will determine whether a given administration is successful and sufficient, or whether subsequent administrations are needed.

Tumor regression and other parameters of successful treatment are assessed by methods known to persons of skill in the art. This includes any imaging techniques that are capable of visualizing cancerous tissues (e.g., MRI), biopsies, methods for assessing metabolites produced by the cancer tissue or affected tissue in question, the subjective well-being of the patient, and the like.

It is also possible to monitor the prognosis of a patient diagnosed with a neoplastic disease. In one embodiment of the present invention, the level of bcl-2 expression was correlated with the sensitivity of the tumor cells to chemotherapeutic drug. Therefore, detecting the level of bcl-2 expression may not only signify an individual who would benefit by the methods of the present invention, but could be used as an indicator of potential prognosis or time to reoccurrence of disease if a standard treatment regimen is followed. In addition, monitoring bcl-2 expression can also be used as an indicator for potential emergence of multiple drug resistance, suggesting a need to change or alter the chemotherapeutic drug and/or drug combination being used.

The following examples are provided merely as illustrative of various of various aspects of the invention and shall not be construed to limit the invention in any way.

### EXAMPLE I

This example demonstrates that expression of connexin significantly increases the sensitivity of cancer cells to chemotherapeutic drugs. This sensitivity has been correlated with the modulation, i.e., an increase, in the expression of bcl-2. The down regulation of bcl-2 expression and the subsequent increased sensitivity to chemotherapeutic agents resulted in an increase in the number of apoptotic cells.

Cell Culture and Drug Treatment

U251 and T98G were originally obtained from the American Type Culture Collection and maintained in DMEM containing 10% fetal calf serum (FCS). U251cx43-216, U251cx43-217 and T98Gcx43-220 are cell lines derived by transfection of parent cells U251 and T98G with cx43 expression vector, while U251N2, U251N23 and T98GN27 are cell lines transfected with control vector (Huang et al., Cancer Res. 58:5089-5096 (1988), incorporated herein by reference). All chemicals described herein were purchased from Sigma (St. Louis, Mo.). Etoposide (VP 16) was prepared as a stock solution of 140 mM in DMSO. Paclitaxel (Tax) was prepared as a 10 mM stock solution in DMSO. Doxorubicin (DOX) was dissolved in DMSO as a 50 mM stock solution. These drugs were diluted 1000-fold before being added to cells. α-Glycyrrhetinic acid (GA) was prepared as 12.5 mM stock solution in DMSO.

Cell Viability.

In vitro viability was determined by the 3-(4,5-dimethylthiazol-2yl)-2,5-diphenyltetrazolium bromide (MTT) calorimetric assay as described in (Huang et al., Cancer Res. 55:5054-5062 (1995)). Cells (1×10³) were plated in triplicate in 96 well microplates overnight and treated with 5 different concentrations of VP16, Tax and DOX, respectively. At day 6 after treatment, cells were stained and processed according to the manufacturer's instruction (Promega, Madison, Wis.). Absorbance values at 570 nm were plotted as a measure of the relative number of cells. Each assay was repeated at least three times. IC₅₀ values were calculated by Litchfield-Wilcoxon's method (Kitazono et al., J. Natl. Cancer Inst. 21:1647-1653 (1999)) and GRAPH PAD PRISM (San Diego, Calif.; Zeng et al., Cancer Res. 59:5964-5967 (1999)). Both methods gave the identical results.

Cell Survival Assay.

The surviving cell fraction was determined by clonogenicity assay as previously described (Huang et al., J. Cell Biol. 133:211-220 (1996)). Cells were seeded at a density of 500 cells per 60 mm plate and incubated overnight and then treated with drugs; culture was then continued for 3 weeks. Clones were fixed in 4% formaldehyde-PBS and stained with Giemsa solution. Colonies containing more than 50 cells were counted and the fraction of surviving cells was calculated. The results represented the average of three separate experiments.

Apoptosis Assays.

Apoptosis was performed by three different methods: Hoechst dye staining, TUNEL assay and annexin V assay.

Hoechst Dye Stain

The assay was performed as we previously described (Huang et al., Cell Death Differ. 5:96-106 (1998)). Briefly, cells were fixed in Carnoy's solution (methanol:glacial acid; 3:1) and stained with 5 μg/ml of bisbenzimide trihydrochloride (Hoechst 33258) for 20 min. The morphology of nuclei was then observed with a Zeiss (Thornwood, N.Y.) photomicroscope II. At least 500 nuclei in each cell line were counted. Experiments were repeated four times.

TUNEL Assay

Cells (1 to 3×10⁵) were seeded onto glass slides in 8-well plates overnight, and then treated with VP16 for 48 hours. Apoptotic cells were then analyzed with a TUNEL-based in situ cell death detection kit (Boehringer-Mannheim, Mannheim, Germany). Fluorescent cells were observed under a fluorescence microscope and viewed as positive cells. The experiment was repeated twice.

Annexin V Assay

Cells, treated or not VP16, were analyzed with the TACS Annexin V-FITC kit (Trevigen, Gaithersburg, Md.) according to manufacturer's instructions. Apoptosis was detected by the appearance of patches of fluorescence on the cell surface.

FACS Analysis

Cell-cycle distribution was determined by flow cytometry as described previously (Huang et al., Int. J. Cancer 77:880-886 (1998)). Briefly, cells were seeded in 100 mm plates (5×10⁵ per plate), incubated for 24 hours, and then treated with VP16 or vehicle. At days 1 and 2 of treatment, cells were collected, fixed in 70% ethanol and stained with propidium iodine. The DNA content of cells was then analyzed in a fluorescence cell sorter (FACSCalibur, Becton Dickinson, Mountain View, Calif.).

Western Blot

The assay was conducted as described by Huang, et al. (Int. J. Cancer 72:102-109 (1997)). Briefly, cell extracts containing equal amounts of protein (about 40 μg) were separated by 10% (for detection of cx43 and β-actin) or 13% (for detection of bcl-2, bax-1, bad-1 and mcl-1) sodium dodecylsulfate-polyacrylamide gel electrophoresis (SDS-PAGE), followed by transferal of proteins onto polyvinylidine difloride membrane (IMMOBILON, Millipore, Bedford. MA). Specific antigens were detected with corresponding antibodies and visualized using an enhanced chemi-luminescence detection kit (Amershan, Aylesbury UK). Anti-cx43 is a polyclonal antibody raised against cx43 synthesized peptides (Hossain et al., J. Cell Physiol. 174:66-77 (1998)). Anti-bcl-2, bax-1, bad-1 and mcl-1 are polyclonal antibodies and have been previously described (Krajewski et al., Cancer Res. 55:44714478 (1995)). Anti-α-actin is a mouse monoclonal antibody purchased from Sigma (Saint Louis, Mo.).

Transfection

U251cx43-216 and U251N23 were transfected with pRC/CMVbcl-2 expressing vector and DsphygroBgl2 using calcium phosphate precipitation (Huang et al., Int. J. Cancer 77:880-886 (1998)). Resistant cells were selected with 30 μg/ml hygromycin B (Calbiochem, La Jolla Calif.) and 400 μg/ml G418. Both hygromycin B- and G418-resistant clones were isolated by colony selection and expanded for the subsequent experiments.

Dye Transfer

Gap junctional communication (GJC) was assayed by transfer of the fluorescent Lucifer yellow (LY) after single-cell micro-injection, as described previously (Huang et al., Cancer Res. 58:5089-5096 (1998)). Cells were observed under a fluorescence-inverted microscope after micro-injection at the given time points, and the number of neighboring cells labeled with fluorescent dye was recorded.

Statistical Analysis

Differences between groups were tested by Student's t-test or the Mann-Whitney test. All p values were two-sided, and those of less than 0.5 were considered statistically significant.

Cx43 Enhances the Cytotoxicity of Chemotherapeutic Agents

The sensitivity of cx43 and control-transfected cells to the cytotoxic effects of VP16 were examined. Following treatment with VP16, cx43-transfected cells showed markedly increased cytopathic effects compared to control-transfected cells treated with an equivalent concentration of VP16 under microscope. To quantitate sensitivity to VP16, two approaches were applied: viability determined by MTT assay and survival fraction determined by clonogenicity assay. In the MTT assay, cells (cx43-transfected and control-transfected) were exposed to VP16 at different concentrations. At day 6 after treatment, viable cells were determined by the MTT assay. Values at OD 570 reflect the relative number of viable cells. As shown in Table 1, the level of toxicity (measured as IC₅₀) induced by VP16 in U251 cx43-216 (cx43-transfected cells) was about 2-fold higher than that of U251N23 (control-transfected cells). In the clonogenicity assay, cells (500 cells/plate) were treated with different concentrations of VP16 and, after 3 weeks, fixed and stained with Giemsa. Clones so formed were counted and compared with untreated cells. As shown in Table 2, cells transfected with cx43 had greatly reduced colony-formation ability following exposure to VP16 compared with control-transfected cells. These studies indicate that, as determined by morphology, survival fraction (clonogenicity assay) and viability (MTT assay), over-expression of cx43 sensitizes U251 cells to cytotoxic effects of VP16.

Since over-expression of cx43 enhanced the sensitivity of U251 cells to VP 16, it was important to determine if cx43 over-expression sensitizes these cells to the cytotoxic effects of other chemotherapeutic agents with different mechanisms of cytotoxicity. As shown in Table 1 and Table 2, over-expression of cx43 also enhanced the cytotoxic effects of Tax, which functions as a tubulin inhibitor, and doxorubicin, which like VP16 inhibits the activity of DNA topoisomerase II, although in a different manner.

The effect of cx43 on the sensitivity was not a clonal variation. Other cx43-transfected clones (U251cx43-217 and T98Gcx43-220) also exhibited enhanced sensitivity to VP16 treatment compared to control-transfected cells (U251N2 and T98GN28) (Table 1 and 2).

TABLE 1
Expression of cx43 Enhances Cytotoxicity of Chemotherapeutic Agents¹

| | |
|---|---|
| IC₅₀ | P Value |

| | | | | | |
|---|---|---|---|---|---|
| VP16 (μM) | Tax (nM) | DOC (nM) | VPI6 | Tax | DOC |

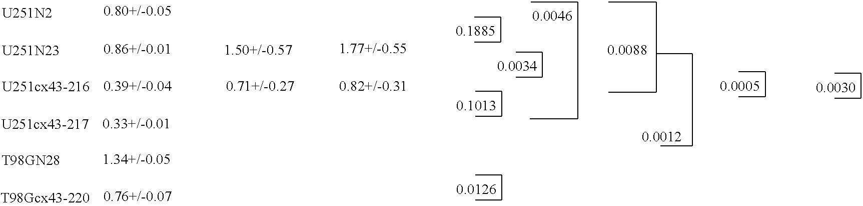

¹Cell survival was determined by MTT assay and reported as the concentrations that inhibits the response by 50% (IC₅₀ ); p values were obtained by Student's t-test; the experiments were repeated at least three times.

TABLE 2
Colony Formation Following Treatment with Chemotherapeutic Agents

| | | U251N23 | U251cx43-216 | P value |
|---|---|---|---|---|
| | — | 180 +/− 36 | 103 +/− 2 | |
| | DMSO | 172 +/− 5 | 98 +/− 3 | |
| VP16 (μM) | 4 × 10⁻⁷ | 129 +/− 16 | 44 +/− 5 | 0.042 |
| | 1 × 10⁻⁷ | 36 +/− 1 | 3 +/− 1 | |
| | 4 × 10⁻⁶ | 9 +/− 1 | 2 +/− 1 | |
| DOX (μM) | 4 × 10⁻¹⁰ | 147 +/− 14 | 70 +/− 6 | 0.024 |
| | 1 × 10⁻⁹ | 15 +/− 5 | 6 +/− 1 | |
| | 4 × 10⁻⁹ | 6 +/− 0 | 2 +/− 3 | |
| Tax (μM) | 4 × 10⁻¹⁰ | 108 +/− 9 | 54 +/− 5 | 0.049 |
| | 1 × 10⁻⁹ | 37 +/− 6 | 9 +/− 5 | |
| | 4 × 10⁻⁹ | 16 +/− 2 | 4 +/− 3 | |

Cell survival in response to chemotherapeutic agents were determined by Clonogenicity assay. After 3 weeks of chemotherapeutic agent treatment, colonies that formed from each sample were counted. At least three independent experiments were done. P values were determined by Mann-Whitney test.

The effect of cx43 on Cytotoxicity is Caused by an Increase in Apoptosis.

It was next determined whether the enhanced sensitivity to chemotherapeutic agents induced by over-expression of cx43 was associated with the increase of drug-induced apoptosis. Cells collected at various time points post-treatment with 1 μM VP16 were used for apoptosis assays. First, nuclear condensation, chromatin fragmentation and formation of apoptotic bodies were detected by Hoechst 33258 dye staining upon treatment with VP16. As shown in Table 3, at day 4 after VP16 treatment, about 29.3% of cells displayed typical apoptotic morphological change, while only 8.8% of control transfected cells underwent apoptosis. The results were further confirmed by both 1) TUNEL assay, which detects double- as well as single-stranded DNA breaks during apoptosis by labeling the free 3′-OH termini in an enzymatic reaction (terminal deoxynucleotidyl transferase), and annexin V assay, which detects the exposed phosphotidylserine during apoptosis. As shown in Table 3, cx43 expression significantly enhanced the sensitivity to VP16 compared to control-transfected cells, though the percentage of apoptotic cells was higher than detected by Hoechst dye staining, reflecting the high sensitivity and detection of earlier events during apoptosis by both TUNEL and annexin V assays.

TABLE 3
Percentage of Apoptotic Cells Induced by VP16

| | Hoechst Stain | | TUNNEL Assay | | Annexin Stain | | | |
|---|---|---|---|---|---|---|---|---|
| | D4 | | D2 | | D2 | | D4 | |
| | — | VP | — | VP | — | VP | — | VP |
| U251N23 | 0.2 +/− 0.2 | 8.8 +/− 2.4 | 0.2 +/− 0.2 | 1.6 +/− 2.3 | 1.0 +/− 0 | 5.5 +/− 0.7 | 2.5 +/− 0.7 | 11.5 +/− 2.1 |
| U251cx43-216 | 2.0 +/− 0.8 | 29.3 +/− 2.4 | 0.2 +/− 0.2 | 13.3 +/− 4.5 | 1.0 +/− 0 | 24.5 +/− 0.7 | 3.5 +/− 0.7 | 42.0 +/− 5.6 |
| P value | 0.1192 | 0.0021 | 0.5000 | 0.0110 | 0.5000 | 0.0007 | 0.1464 | 0.0095 |

Experiments were performed at least three times. P values were determined by student's t test between U251N23 and U251cx43-216.

Since cx43-transfected cells grow slower than control-transfected cells, one explanation of the different sensitivities to VP 16 is the different cell number between cx43-transfected and control-transfected cells. To rule out this possibility, cx43-transfected cells were seeded at a two-fold higher density (2×10⁵ per 60 mm plate) than the routine seeding density (1×10⁵ per 60 mm plate) and an apoptosis assay was performed upon exposure to 1 μM VP16. Data demonstrated that VP16 treatment did not change the percentage of apoptotic cells between high and low cell density in cx43-transfected cells, suggesting that the cell number was not the limiting factor in this system.

The effect of cx43 on increased cytotoxicity by paclitaxel was also caused by apoptosis. Cx43-transfected cells (U251cx43-216 and U251cx43-217) and control-transfected cells (U251N2 and U251N23) were treated with different concentrations of Tax (0, 10⁻⁹, and 4×10⁻⁹). Four days after treatment, cells were analyzed for apoptosis by Hoechst dye staining. Cx43-expressing cells exhibit a 3- to 4-fold increase in apoptosis compared with control-transfected cells. When cx43-transfected cells were treated with 4×10⁻⁹ M of Tax, about 65% of cells displayed typical features of apoptosis, while at the same concentration, only about 17% of control-transfected cells were apoptotic.

These results suggest that the constitutive expression of cx43 may play a role in the enhancement of apoptosis by chemotherapeutic agents.

Cx43 Mediated Apoptosis in Response to VP16 Without Modulating G2 Phase Distribution.

Since VP16 treatment lead to G₂ arrest in other cells and p53 was found to enhance sensitivity of VP16 in M1 myeloid leukemia cells by facilitating the G₂ to M transition (Anderson and Roberge, Cell Growth Differ. 7:83-90 (1996); Skladanowski and Larsen, Cancer Res. 57:818-823 (1997)), the enhancement of cytotoxicity to VP16 by cx43 could reflect an effect of cx43 on the VP16-induced G2 arrest. The possibility was assessed by treatment of the cells with VP16 for 24 and 48 hours, and cell-cycle distribution was then determined by FACScan. As shown in Table 4, treatment with VP16 almost completely blocked the cells at G₂ phase, but there was no significant difference of G₂ phase distribution between cx43- and control-transfected cells. Although there was a slight decrease of S phase in cx43-transfected cells compared with control transfected cells at 24 hours after VP 16 treatment, it was not statistically significant. These results indicate that cx43 has no major effect on the cell-cycle progression, especially in G₂ phase in response to VP16.

TABLE 4
Cell cycle distribution in response to VP16

| | | U251N23 | | U251cx43-216 | | U251cx43-217 | |
|---|---|---|---|---|---|---|---|
| treatment | day | phase | % | phase | % | phase | % |
| — | 1 | G1 | 57.24 | G1 | 70.30 | G1 | 70.00 |
| | | S | 27.71 | S | 21.66 | S | 18.53 |
| | | G2 | 15.05 | G2 | 10.04 | G2 | 11.47 |
| VP16 | 1 | G1 | 7.37 | G1 | 10.95 | G1 | 12.05 |
| | | S | 11.14 | S | 8.48 | S | 10.11 |
| | | G2 | 81.49 | G2 | 80.57 | G2 | 77.84 |
| — | 2 | G1 | 60.19 | G1 | 72.82 | G1 | 73.44 |
| | | S | 22.81 | S | 16.11 | S | 17.10 |
| | | G2 | 17.00 | G2 | 11.07 | G2 | 9.46 |
| VP16 | 2 | G1 | 5.78 | G1 | 8.44 | G1 | 11.39 |
| | | S | 0 | S | 0.75 | S | 0 |
| | | G2 | 94.22 | G2 | 90.81 | G2 | 88.61 |

Log phase cells at day 2 after seeding were exposed to VP16 (1 × 10⁻⁶ M). At day 1 and day 2 after treatment, cells were harvested for the determination of cell cycle distribution by FACScan. The experiments were repeated once and similar results were obtained.

Regulation of Expression of Apoptosis-Related Genes by cx43.

The balance between apoptosis-protecting genes such as bcl-2, bcl-x, mcl-1 and bag-1, and apoptosis-promoting genes such as bax-1, A1, bad-1 and p53, regulates apoptosis. To examine whether the cx43-mediated apoptosis in response to VP16 was linked to the expression of apoptosis-related genes, the expression of some of these genes was examined by Western blot analysis. Both cx43- and control-transfected cells were treated with or without VP16 for 48 hours or grown under low serum conditions (0.2% CS) for 6 days. Cell lysates containing equal amounts of total protein were subjected to Western blot analysis using antibodies against bcl-2 and β-actin (as loading control). Bcl-2 expression was significantly reduced in cx43-transfected cells. Quantitation by densitomitry reveals that bcl-2 levels were reduced about 6- to 8-fold in cx43-transfected cells. The expression of other apoptosis-related genes such as bax-1, bad-1, bcl-x_{L} and mcl-1 did not change.

Elevation of bcl-2 Levels Partially Reduces Apoptosis in Response to VP16 in cx43-Transfected Cells.

The down-regulation of bcl-2 in cx43-transfected cells raised the question of whether increased apoptosis in cx43-transfected cells in response to chemotherapeutic agents was mediated by the reduction of bcl-2 expression. To test this possibility, a bcl-2 expression vector was transfected into U251cx43-216 and U251N23 cell lines together with a hygromycin expression vector. Several clones expressing high amount of bcl-2 levels were then identified by Western blot analysis.

The effect of bcl-2 on apoptosis in response to VP16 was then examined using these bcl-2 over-expressing cell lines and hygromycin control-transfected cell lines. Cells were then treated with 10⁻⁶ M of VP16 for 4 days and assayed for apoptosis by Hoechst dye staining. Expression of bcl-2 in cx43-transfected cells (UCBm and UCB5) significantly reduced apoptosis in response to VP16 compared with the control cells (U251cx43-216 and UCN22). Furthermore, bcl-2 expression in cx43-transfected cells profoundly increased the colony-formation frequency compared with control-transfected cell. These results suggested that bcl-2 was one of the major targets of cx43 and that reduced bcl-2 expression in cx43-transfected cells at least partially contributed to the increased apoptosis in cx43-transfected cells. However, there was still more apoptosis in cells expressing both cx43 and bcl-2 than in cells expressing bcl-2 alone, suggesting that additional mechanisms were operating in cx43-transfected cells in response to chemotherapeutic drugs.

Cx43-Mediated Apoptosis is Independent of the Gap Junction Communication.

The results presented above clearly demonstrate that cx43 expression enhanced VP16-induced apoptosis in human glioblastoma cells. It was next determined whether cx43-mediated apoptosis was related to gap junctional communication (GJC). Previously, it had been demonstrated that cx43 expression did not increase GJC in U251 and T98G cells as measured by transfer of fluorescent Lucifer yellow dye into the neighboring cells. Since Lucifer yellow dye transfer may not accurately reflect the ability of intracellular substances to pass through gap junctions, apoptosis was examined in response to VP16 in the presence of α-Glycyrrhetinic acid (GA), which inhibits GJC (Davidson et al., Biochem. Biophys. Res. Commun. 134:29-36 (1986); Davidson and Baumgarten, J. Pharmacol. Exp. Ther. 266:1104-1107 (1988)). No significant alteration of apoptosis in the presence of α-Glycyrrhetinic acid was found in either cx43- or control-transfected cells. Consistent with previous studies, both cx43- and control-transfected cells poorly communicated each other as examined by Lucifer yellow dye transfer experiment. The effect of GA was not obvious due to poor GJC in untreated U251 cells. However, GA completely blocked GJC in T51B cells, suggesting that GA has a potent effect on GJC (Table 5). Thus, the difference in apoptosis is probably due to the presence of cx43 itself rather than GJC. Furthermore, cx43-transfected cells expressed only non-phosphorylated cx43 in both mock- and VP 16-treated cells. In contrast, when the same experimental procedure was used to detect cx43 in primary astrocytes and T51B cells, several phosphorylated isoforms were clearly observed (Huang et al., Cancer Res. 58:5089-5096 (1998)). It is generally believed that phosphorylated forms of cx43 are involved in GJC (Hossain et al., J. Cell Physiol. 174:66-77 (1998)). Therefore it is likely that cx43-mediated apoptosis in response to VP16 is not related to its gap junctional communication effect.

TABLE 5
Gap Junction Communication in the Presence of GA

| | No. of Fluorescent Neighboring cells after GA (25 μM) treatment¹ | | | | |
|---|---|---|---|---|---|
| Cells | — | 5 min | 24 h | 48 h | 72 h |
| U251N23 | 2.7 +/− 2.7 | 0.7 +/− 1.1 | 2.2 +/− 3.7 | 0.9 +/− 1.9 | 2.4 +/− 4.1 |
| U251cx43-216 | 1.5 +/− 1.8 | 0.6 +/− 1.3 | 0 +/− 0 | 0.6 +/− 1.1 | 0.1 +/− 0.3 |
| T51B² | 81.2 +/− 6.0 | 0 +/− 0 | | | |

¹Gap junction communication was determined by the number of fluorescent neighboring cells after injection of LY into single cells. The values are the average of from 12 to 20 injections as indicated in parentheses.
²T51B are rat liver epithelial cells with good communication ability and used here as positive control.

The data provided herein suggests that cx43 functions as a tumor-suppressor gene. Since other tumor-suppressor genes, such as p53, can sensitize cells to apoptosis in response to chemotherapeutic drugs, whether cx43 expression in human glioblastoma cells was able to enhance to sensitivity of tumor cells to chemotherapeutic agents was examined. It was found that human glioblastoma cells expressing cx43 became more sensitive to cytotoxicity to several chemotherapeutic drugs used at clinically relevant concentrations. The drugs to which cx43-expressing cells displayed increased sensitivity have diverse mechanisms of action and included (i) a topoisomerase II inhibitor (etoposide, VP16); (ii) paclitaxel (Tax), which inhibits microtubulin assembly; and (iii) doxorubicin, another topoisomerase inhibitor that acts in a different way from VP16. These findings suggest that cx43 functions in a relatively distal common pathway for cell death induced by multiple mechanisms.

Over-expression of cx43 decreases expression of the bcl-2 protein and significantly enhanced cell death during exposure of cells to chemotherapeutic drugs. Based on this finding, it has been predicted that patients having glioblastoma containing low levels of cx43 and high levels of bcl-2 will have a poor prognosis compared to those who present with histologically and clinically similar disease, but whose neoplasm expresses high levels of cx43 and low levels of bcl-2. Several reports suggest that the decreased bcl-2 levels are indeed associated with shorter disease-free survival in human glioblastoma (Deininger et al., Cancer 86:1832-1839 (1999); Newcomb et al., Acta Neuropathol. 94:369-375 (1997)).

The mechanisms responsible for cx43-mediated apoptosis in response to chemotherapeutic drugs are unknown. Since cx43 is the structural component of gap junctions responsible for the transfer of water-soluble molecules directly from one cell to another without passing through the membrane, the enhancement of cytotoxic effects on cx43-transfected cell may be due to increased transfer of drugs from one cell to another, especially when the molecular weight of the drugs is less than 1 kDa. Indeed, thioguanine-derived nucleotides were presumably transferred from HPRT⁺ (hypoxanthine-guanine phosphoribosyltransferase) to HPRT⁻ cells to kill those HPRT⁺ contacting HPRT⁻ cells through GJC (Fujimoto et al., Proc. Natl. Acad. Sci. USA 68:1516-1519 (1971)). Recent studies also suggest that the bystander effect seen in HSV-tk gene therapy may be due to connexin-mediated GJC (Mesnil et al., Proc. Natl. Acad. Sci. USA 93:1831-1835 (1996)). However, in the experiments presented herein, several lines of evidence do not support a role for intercellular communication in mediating apoptosis: (i) a significant increase in GJC in cx43-transfected cells was not observed; (ii) cx43-transfected cells predominantly expressed the non-phosphorylated form of cx43 in the presence or absence of VP16; (iii) when a potent and long-term inhibitor of GJC, α-Glycyrrhetinic acid, was added during treatment of VP 16, no decrease of apoptosis was observed; (iv) in the clonogenicity assays, cells were sparsely seeded (e.g., 500 cells per 60 mm plates) so that they were not in contact and not able to form GJC. It can be concluded from these data that the enhanced sensitivity to chemotherapeutic drugs by cx43 must be due to cx43 action that is not directly related to GJC.

Chen et al., (Cell Growth Differ. 6:681-690 (1995) demonstrated that expression of cx43 in dog kidney neoplastic epithelial cells, TRMP, altered a set of cell cycle-related gene expressions, suggesting that at least some of cx43 functions are mediated by regulation of downstream gene expression. Lecanda et al., (Mol. Cell. Biol. 9:2249-2258 (1998) also reported that expression of cx43 modulates gene expression in osteoblastic cells. Therefore, the expression of several apoptosis-related genes was examined. Among them bcl-2 expression was specifically reduced in cx43-transfected cells. The expression of bax-1, bad-1, mcl-1 and bcl-x_{L} was not changed. Since U251 cells express mutant p53, the cx43-mediated apoptosis in response to VP16 does not require wild-type p53 function. Thus, one of the mechanisms responsible for the cx43-mediated apoptosis may be due to regulation of bcl-2 expression. Indeed, gene-transfection experiments suggest that the cx43-mediated apoptosis in response to chemotherapeutic drugs at least is partially mediated by down-regulation of bcl-2 expression. It is well known that elevated levels of bcl-2 protein in gene-transfection experiments leads to an increased resistance to a wide variety of chemotherapeutic drugs as well as radiation (Miyashita and Reed, Blood 81:151-157 (1993); Piche et al., Cancer Res. 58:2134-2140 (1998); Reed et al., J. Cell Biochem. 60:23-32 (1996)). The mechanism for the regulation of bcl-2 expression is currently unknown. Considering the fact that transfected cx43 was predominantly localized in the nucleus (Huang et al., Cancer Res. 58:5089-5096 (1998)), it is possible that cx43 may directly regulate gene expression through binding to cis elements in the promoter regions of regulated genes. Indeed, it has been reported that cx43 is localized in the nucleus and can bind to DNA, suggesting that cx43 has distinct functions from its well known GJC (de Feijiter et al., Mol. Carcinogenesis 16:203-212 (1996). Alternatively, down-regulation of bcl-2 may result from signal transduction through secondary, downstream elements since cx43 exhibits SH2 and SH3 as well as ZO1 binding sites (Guerrier et al., J. Cell Sci. 108:2609-2617 (1995); Kanemitsu et al., J. Biol. Chem. 272:22824-22831 (1997); Loo et al., Mol. Carcinogenesis 25:187-195 (1999)).

Lin et al., (Nat. Med. 1:494-500 (1998)) reported that gap junctions achieved by transfection of cx43 can mediate the propagation of a death signal between dying and healthy glial cells in a co-culture system. However, in a homogenous culture system, the sensitivity to injury was not simply dependent on the gap junctions. Rather, high levels of bcl-2 protected cells from apoptosis in response to injury, supporting the conclusions of the present invention that down-regulation of bcl-2 may be responsible for the drug-induced apoptosis in cx43-transfected cells.

The effect of cx43 on the enhanced sensitivity to chemotherapeutic drugs could also result from an increase in the retention or a decrease in the elimination of the drugs. The results provided herein clearly demonstrate a role for cx43 in chemotherapeutic drug-induced apoptosis. In addition, human glioblastoma tumors transfected with cx43 demonstrate down-regulation of bcl-2 and increased apoptosis. This effect of cx43 is not mediated by gap junction communication (GJC), thus demonstrating additional functions of this protein.

### EXAMPLE II

This example simultaneously examined the presence or absence of forty three (43) cytokines, chemokines and growth factors in cx43-transfected and non-transfected cells. Examples of cytokines, chemokines and growth factors included MCP-1, IL-10, IL12, IL-13, IL-15, IFN-γ, GCSF, IGF-1, TGF-β1, TNFα, VEGF and the like. MCP-1 was demonstrated to be down regulated in cx43-transfected cells.

Materials

All pair antibodies were purchased either from BD PharMingen (San Diego, Calif.) or from R&D (Minneapolis, Minn.). Cytokines were obtained from Propetech (Rocky Hill, N.J.), BD PharMingen and R&D. Horse-Radish Peroxidase-(HRP)-conjugated streptavidin was purchased from BD PharMingen. Cy3-conjugated streptavidin was the product of Rockland (Gilbertsville, Pa.).

Preparation of Array Membranes

The preparation of array membranes was as described in (Huang, J Immunol. Methods 255:1-13 (2001); Huang, et al., Anal. Biochem. 294:55-62 (2001)). Briefly, a computed generated-template was used to guide to spot solution onto membranes. 0.20 μl of capture antibodies (200 μg/ml) were manually loaded onto membranes by a 2 μl pipeman in duplicate. HRP-conjugated antibody was spotted onto membranes as positive control and identification of orientation of arrays.

Human Cytokine Chip Technology

300 pL of capture antibodies (500 μg/ml) were printed onto Hydrogel chips (Packard Bioscience, Meriden, Conn.) using the Biochip Arrayer (Packard Bioscience). After blocking, the chips were incubated with 50 μl of different samples, including non-transfected, control transfected (U251N23) and transfected (U251cx43-216) cells at room temperature for 2 hr. The chips were then washed with to remove unbound components. Biotin-labeled detection antibody cocktail was added (50 μl/chip) and incubated at room temperature for 1 hr. After wash, Cy3 labeled streptavidin was added and the chips were incubated at room temperature for 1 hr. The excess amount of Cy3 streptavidin was removed and the signals were scanned by laser scanner (Affymetrix, Santa Clara, Calif.). A series of diluted Cy3 streptavidin, Cy5 streptavidin and Biotin IgG (BIgG) were included as positive control. BSA was used as negative control.

Immuno-Western Blot Analysis

Immuno-Western blot was carried out as described (Huang, et al., J. Cell Biol. 133:211-210 (1996); Huang, et al., Mol. Carcinog. 30:209-217 (2001)). Essentially, cells were seeded at a density of 1×¹⁰⁻⁶ per 100-mm dish. After 48 hrs conditioned media was collected. Nonconcentrated medium 1×) or 10 fold concentrated medium (10×) were incubated with anti-MCP-1 at 4° C. for 2 hr. The antigen-antibody complex was precipitated by Staphylococcus aureus. The precipitated complex was analyzed by SDS-PAGE. After transferring the protein to membranes, the presence of MCP-1 was detected by anti-MCP coupled with ECL system.

Reverse Transcription-PCR

RT-PCT was performed according to (Huang, et al., Cancer Res. 58:5089-5096 (1998)). Briefly, total RNA was isolated from culture cells by the guanidine isothiocyanate RNAzolB method (Cinna/Biotecx Laboratories, Houston, Tex.). 5 μg of total RNA was used for cDNA synthesis using random hexamer primer (Boehringer Mannheim, Germany). PCR amplification was carried out by using all of reverse-transcribed RNA. The PCR reaction mixture contained 50 mM KCl; 2.5 mM MgCl₂; 10 mM Tris pH 8.0; 10 mM DNTP; 10 μM of each primer and 0.5 unit of Taq polymerase (Boehringer Mannheim) in the final volume of 50 μl. The PCR profile was 94° C. for 40 s, 52° C. for 50 s, and 72° C. for 60 s for 25 cycles, followed by 75° C. for 5 min. After PCR, the input RNA was removed by RNase digestion. The amplified DNA was then precipitated and separated on 1.8% agarose gel containing ethidium bromide. The sense primer was 5′CAA ACT GAA GCT CGC ACT CTC GCC 3′ (SEQ ID NO. 1). The antisense primer was 5′ GCA AAG ACC CTC AAA ACA TCC CAG G 3′ (SEQ ID NO: 2). The expected amplified fragment of human MCP-1 was 327 bp. As an internal control, β-actin primes were used as previously described (King et al., Carcinogenesis 21:311-315 (2000)) to detect 245 bp of β-actin product.

cDNA Microarrays

Assays were done according to manufacturer's instruction. Briefly, two Atlas human cDNA expression array membranes were purchased from Clontech (Palo Alto, Calif.). 5 μg of mRNA isolated from cx43-transfected cells (U251cx43-216) and control-transfected cells (U251N23) were then treated with DNase I and first-strand cDNA synthesis was carried out in the presence of ³²P dATP. Equal amounts of cDNA from cx43-transfected and control-transfected cells were then hybridized to two identical Atlas human cDNA expression arrays in separate bags. The expression arrays were washed. The image was obtained by exposure to X-ray film and phosphoimager.

³H-Thymidine Incorporation Assay

The experiment was performed as described in Huang et al., (Cancer Res. 55:5054-5062 (1995); and Oncogene 10:467475 (1995)). Briefly, cells were seeded in 96-well plates. 24 hr later, cells were incubated in the presence of cytokine or conditioned medium for 48 hours. 0.5 μCi of ³H-thymidine was added to each well and incubation was continuous for 24 hr. The incorporated ³H-thymidine was then determined by a scintillation counter.

CyQUANT Cell Proliferation Assay

The assay was carried out according to the manufacturer's instruction (Molecular Probe, Eugene Oreg.). Briefly, 1,000 cells were seeded in 96 well plates. 24 hrs. later, different concentrations of antibody were added to tissue culture cells. The plates were incubated at 37° C. for another 48 hrs. Cell number was determined by incubation with CyQUANT dye and the fluorescence was measured using a CCD imaging system (Bio-Rad, Hercules, Calif.) with filters for 480 nm excitation and 520 nm emission.

Soft Agar Assay

Soft agar assay were performed as described previously (Huang, et al., Cancer Res. 58:5089-5096 (1998); Huang, et al., Mol. Carcinog. 30:209-217 (2001); Huang, et al., Carcinogenesis 20:485492 (1999)). Briefly, cx43-transfected cells and control-transfected cells were assayed by seeding 1,000 cells in 0.26% agar medium into 6 well plates previously lined with 0.65% agar medium. The plates (in duplicate and repeated twice) were cultured for 3-4 weeks in the presence of different treatments and then stained with p-iodotetrazolium violet for overnight before photography and counting. Colony size equal to or greater than 15,625 μm² was scored as positive.

Identification of cx43 Regulated Cytokines by Human Cytokine Array System

The potential cx43-regulated cytokines in cx43-transfected and control-transfected cells were screened as described above with the array. Expression of MCP-1 was significantly reduced in cx43-transfected cells. All other cytokines were similar between cx43-transfected and control-transfected cells. To further confirm the human cytokine array results, immunoprecipitation of conditioned media from cx43-transfected cells and control-transfected cells were performed with antibody against MCP-1. The immunoprecipitated complex was then separated by SDS PAGE and the levels of MCP-1 protein were detected by Western Blot using antibody against MCP-1. MCP-1 was prominently expressed in conditioned media from control-transfected cells (U251N23), but not from cx43-transfected cells U251cx43216).

To examine whether the down-regulation of MCP-1 expression was mediated by transcription regulation, semi-quantitative RT-PCR was applied to measure MCP-1 mRNA levels. MCP-1 was only detected in U251N23 cells. To make sure that this result did not simply reflect clonal variation, the expression of MCP-1 in other cx43-transfected cells (U251cx43-217) and other control-transfected cells (U251N2) was expressed. Again, MCP-1 was highly expressed in the control-transfected cells but not in cx43-transfected cells.

Analysis of cx43 Regulated Genes By cDNA Microarrays

To further exploit the molecular mechanisms responsible for the cx43-mediated tumor suppression, an Atlas human cDNA microarray system was applied. 5 μg of mRNA prepared from U251cx43-216 cells (human glioblastoma cells transfected with cx43 expression vector) and U251N23 (human glioblastoma cells transfected with control vector) were used to generate ³²P-labeled cDNA microarray probes. Probes derived from each transfected cell line were hybridized to Atlas human cDNA expression array membranes containing 588 human cDNA. To avoid variability created by the striping process, hybridization was performed on two membranes with U251cx43-216 probe and U251N23 probe, respectively. Each hybridization membrane was exposed to X-ray film and scanned with a phosphorimager.

The quantification of the Atlas human cDNA expression array membrane was performed using a computer program in AWK Script running under the Unix environment to automate the comparison procedure. The intensity of signal in the membranes was calculated by Image QuaNT program (Molecular Dynamics). The quantitative scores were normalized using the scores of actin spotted on the same membrane. To identify genes, which were up regulated or down regulated in cx43-transfected cells, the ratios of the sum scores (minus background) between U251cx43-216 and U251N23 were calculated for each spot. Since each cDNA was spotted in duplicate, there are two spots for each cDNA and thus two ratios. Those cDNAs, which are consistent across both ratios, were considered to be genuinely regulated in U251cx43-216 cells. A ratio of more than 2 was taken as cut-off score to access if a gene is up- or down-regulated. According to this standard, monocyte chemotactic protein-1 (MCP-1) was found to be specifically down-regulated in cx43-transfected cells (more than 5 fold reduction in cx43-transfected cells).

Down-Regulation of MCP-1 by cx43 is Involved in Cell Proliferation

The down-regulation of MCP-1 in cx43-transfected cells raised the question of whether reversion of the transformed phenotype in cx43-transfected cell was mediated by the reduction of MCP-1 expression. To test this possibility, anti-MCP-1 neutralization antibody was added into the tissue culture medium to block the MCP-1 activity and to examine the cell proliferation by CyQUANT cell proliferation assay. Addition of anti-MCP-1 antibody significantly inhibited the cell proliferation rate in U251 cells transfected with control vector, U251N23, which expressed high amount of MCP-1, but not in cx43 transfected cells, which accumulated very low level of MCP-1. In contrast, U251N23 cell conditioned medium specifically enhanced cell proliferation rate in cx43-transfected cells. Furthermore, addition of MCP-1 specifically stimulated cell proliferation rates in cx43-transfected cells but not in control-transfected cells, suggesting the involvement of MCP-1 might be one important factor contributing to cell growth control in human glioblastoma cells.

To examine the effect of down-regulation of MCP-1 on the transformed growth, cx43-transfected and control-transfected clones were assayed for their anchorage-independent growth in soft agar in the presence of MCP-1. Table 6 showed that addition of MCP-1 increased colony formation of cx43-transfected cells in soft agar.

TABLE 6
Effect of MCP-1 on Colony Formation in Soft Agar

| | | U251N23 | U251N2 | U251cx43-216 | U251cx43-217 |
|---|---|---|---|---|---|
| MCP-1 (ng/ml) | Control | 36.0 ± 5.65 | 24.5 ± 0.70 | 4.0 ± 1.41 | 7.0 ± 1.41 |
| | 0.1 | | | 18.0 ± 2.83 | 18.0 ± 2.83 |
| | 1 | | | 37.5 ± 3.53 | 24.5 ± 2.12 |
| | 10 | | | 28.5 ± 4.95 | 22.5 ± 3.53 |
| | 100 | | | 36.0 ± 5.65 | 20.5 ± 2.12 |

Colonies containing > ˜100 cells (i.e. ˜200 μm diameter) were scored positive.

Cx43-transfected clones and control-transfected clones were assayed for apoptosis in the presence of MCP-1 or anti-MCP-1 antibody. Addition of MCP-1 or anti-MCP-1 antibody did not have any effect on the apoptosis under normal culture conditions, or under low serum conditions or in response to chemotherapeutic drugs. Therefore, enhanced apoptosis under low serum conditions and decreased cell growth in cx43-transfected cells involves at least two separate pathways.

Growing evidence suggests that cx43 functions as a tumor suppressor gene. However, the molecular mechanisms involved in tumor suppression are still ill defined. To determine whether secreted factors contribute to tumor suppression by c43, a human cytokine array system has been developed which allows simultaneous detection of 43 cytokines, chemokines and growth factors.

MCP-1 was found to be down-regulated in cx43-transfected cells. This conclusion was further confirmed by immunoprecipitation analysis, RT-PCR, cDNA microarray and enhanced protein arrays (data not shown). A wealth of evidence suggests that MCP-1 may play an important role in tumorigenesis. In contrast to the majority of normal cells, many human and murine tumor cells were shown to constitutively produce high levels of MCP-1, including human glioblastoma (Desbailets, et al., Int. J. Cancer 58:240-247 (1994)), melanoma (Nesbit, et al., J. Immunol. 6483-6490 (2001)), ovarian cancer (Hefler, et al., Bio. J. Cancer 81:855-859 (1999)), breast carcinoma (Wong, et al., J. Pathol. 186:372-377 (1998)), Hodgkin' disease (Luciani, et al., Mol. Pathol. 51:273-276 (1998)) and lung cancer (Wong, et al., J. Pathol. 186:372-377 (1998)). Clinical studies suggested that high expression of MCP-1 was a significant indicator of early relapse of human breast cancer (Ueno, et al., Clin. Cancer Res. 6:3282-3289 (2000)). MCP-1 expression has also been suggested to contribute to the high malignancy phenotype of murine mammary adenocarcinoma cells (Neumark, et al., Immunol. Lett. 68:141-146 (1999)). In addition, MCP-1 has been demonstrated to be capable of inducing angiogenesis, which is a critical event for tumor growth (Nesbit, et al., J. Immunol. 166:6483-6490 (2001); Goede, et al., Int. J. Cancer 82:765-770 (1999)). Expression of MCP-1 has also been tightly associated with chronic inflammation, which may promote tumor development (Dong, et al., J. Interferon Cytokine Res. 18:629-638 (1998); deBoer, et al., J. Pathol. 190:619-626 (2000)). cDNA microarray technology revealed the association between the development of drug resistance in ovarian cancer cells and the accumulation of MCP-1 {Duan, et al., Clin. Cancer Res. 5:3445-3453 (1999)). Furthermore, other chemokines or chemokine receptors such as RANTES, CXCR2 and CXCR4 have been shown to be associated with the tumor development (Luboshits, et al., Cancer Res. 59:4681-4687 (1999)).

Considering the possible role of MCP-1 in tumor development, the present results suggests that down-regulation of MCP-1 in cx43-transfected cells contributed to the reversion of tumor cell growth. This hypothesis was tested by several experiments. Addition of anti-MCP-1 antibody to tissue culture media of control-transfected cells but not in cx43-transfected cells. In contrast, MCP-1 and conditioned medium from control-transfected cells promoted cx43-transfected cell growth both in monolayer and in soft agar. The role of MCP-1 on the human glioblastoma cell growth therefore is likely mediated through an autocrine mechanism since both cx43-transfected cells and control-transfected cells expressed MCP-1 receptor, CCR2. Consistent with the notion that MCP-1 is one of major targets in the control of human glioblastoma cell growth, the expression of cx43 has been previously shown to be decreased in several human glioblastoma cell lines and patient surgical tumor tissues (Huang, et al., J. Surg. Oncol. 70:21-24 (1999); Huang, et al., Cancer Res. 54:5089-5096 (1998)).

Production of MCP-1 in glioblastoma cells is also responsible for infiltrating macrophages and monocytes. It is well known that tumor-associated macrophages represent one of the first lines of immunological defense against neoplastic cell growth. Therefore, the tumor growth in vivo must be regulated by the balance between stimulation of tumor cell growth by MCP-1 and the MCP-1-mediated macrophage.

All publications and patents mentioned in this specification are indicative of the level of skill of those skilled in the art to which this invention pertains. All publications and patents are herein incorporated by reference to the same extent as if each individual publication or patent was specifically and individually indicated to be incorporated herein by reference.

Although the foregoing invention has been described in some detail by way of illustration and example for purposes of clarity of understanding, it will be obvious that certain changes and modifications may be practiced within the scope of the appended claims.

What is claimed is:

1. A method for inhibiting the proliferation of tumor cells in a mammal, comprising: contacting the tumor cells with a nucleic acid encoding a connexin protein, fragment, derivative, or analog thereof in an amount sufficient to effectivly reduce the expression of bcl-2; and an effective concentration of a chemotherapeutic drug.

2. The method according to claim 1, wherein the nucleic acid encodes a connexin, fragment, derivative or analog, wherein the connexin is connexin 26, connexin 32, connexin 43, or connexin 45.

3. The method according to claim 2, wherein the nucleic acid encodes connexin 43, or a fragment, derivative, or analog thereof.

4. The method according to claim 1, wherein the chemotherapeutic drug is etoposide, paclitaxel, or doxorubicin.

5. The method according to claim 1, wherein the tumor cells from a carcinoma, sarcoma, lymphoma, leukemia, or melanoma.

6. The method according to claim 5, wherein the tumor cells are glioblastoma cells.

7. The method according to claim 1, wherein the nucleic acid is formulated for administration by direct injection, microparticle bombardment, liposome, targeted liposome, microparticle or microcapsule.

8. The method of claim 7, wherein the nucleic acid is incorporated in a recombinant retroviral or adenoassociated viral vector.

9. The method of claim 7, wherein the nucleic acid is formulated as a nucleic acid-ligand complex.

10. The method of claim 1 further comprising administering an antagonist of MCP-1 activity.

11. The method of claim 10, wherein the antagonist of MCP-1 activity is an antibody specific for MCP-1 or a receptor of MCP-1.

12. The method of claim 11, wherein the antibody is a polyclonal or monoclonal antibody or an antigen binding fragment thereof.

13. The method of claim 12, wherein the antibody is a chimeric antibody, a single chain antibody, or a antigen binding fragment thereof.

14 A method for inhibiting the proliferation of tumor cells in a mammal, comprising:
a) contacting the cells with a connexin protein, fragment, derivative, or analog thereof effective to reduce the expression of bcl-2; and
b) contacting the cells with an effective concentration of a chemotherapeutic drug.

15 The method according to claim 14 wherein the connexin protein, fragment, derivative, or analog is derived from connexin 26, connexin 32, connexin 43, or connexin 45.

16. The method according to claim 15, wherein the connexin is connexin 43, or a fragment, derivative, or analog thereof.

17. The method according to claim 14, wherein the chemotherapeutic drug is etoposide, paclitaxel, or doxorubicin.

18. The method according to claim 14, wherein the tumor cells from a carcinoma, sarcoma, lymphoma, leukemia, or melanoma.

19. The method according to claim 18, wherein the tumor cells are glioblastoma cells.

20. The method according to claim 14, wherein the connexin is formulated for administration by direct injection, liposome, targeted liposome, microparticle or microcapsule.

21. The method of claim 14 further comprising administering an antagonist of MCP-1 activity.

22. The method of claim 21, wherein the antagonist of MCP-1 activity is an antibody specific for MCP-1 or a receptor of MCP-1.

23. The method of claim 22, wherein the antibody is a polyclonal or monoclonal antibody or an antigen binding fragment thereof.

24. The method of claim 22, wherein the antibody is a chimeric antibody, a single chain antibody, or a antigen binding fragment thereof.

25. A method of inhibiting the proliferation of a population of target cells in a subject comprising administering to the subject an amount of a connexin protein, fragment, derivative or analog thereof effective to reduce the expression of bcl-2 in combination with an effective amount of a chemotherapeutic drug.

26. The method of claim 25, wherein the connexin protein, fragment, derivative, or analog thereof is connexin 26, connexin 32, connexin 43, or connexin 45.

27. A method of monitoring the prognosis or treatment of a subject undergoing chemotherapy, comprising:
a) isolating a population of tumor cells from the subject;
b) determining the expression level of connexin in the isolated population of cells;
c) determining the expression level of bcl-2 in the isolated population of cells;
d) determining the ratio of the expression level of connexin to the expression level of bcl-2;
e) correlating a better prognosis for the subject with a high ratio of connexin expression when compared to the expression of bcl-2.

28. The method of claim 27, wherein the expression level of connexin and bcl-2 are determined by immunoassay.

29. The method of claim 27, wherein the expression level of connexin and bcl-2 are determined by nucleic acid hybridization.
